(19) United States
(12) Patent Application Publication
(10) Pub. No.: US 2024/0399085 A1
(43) Pub. Date: Dec. 5, 2024

(54) INTELLIGENT RESPIRATORY ENTRAINMENT

(71) Applicant: ResMed Digital Health Inc., San Diego, CA (US)

(72) Inventors: Redmond Shouldice, Dublin (IE); Hannah Meriel Kilroy, Dublin (IE); Kieran Edward McNamara, Dublin (IE)

(21) Appl. No.: 18/687,787

(22) PCT Filed: Aug. 30, 2022
(86) PCT No.: PCT/IB2022/058134

§ 371 Date: Feb. 28, 2024

Related U.S. Application Data
(60) Provisional application No. 63/238,410, filed on Aug. 30, 2021.

(51) Int. Cl. A61M 16/00; A61M 21/00; A61M 21/02

(52) U.S. Cl. CPC A61M 16/024; A61M 21/02; A61M 2021/0011; A61M 2021/0022; A61M 2021/0027; A61M 2021/0044; A61M 2021/0088; A61M 2205/581; A61M 2205/582; A61M 2205/583; A61M 2230/42

(57) ABSTRACT

An intelligent entrainment program can make use of received biometric sensor data to provide hyper-personalized guidance to entrain a user's respiration pattern towards a target respiration pattern. The entrainment program can be used for sleep-related therapy, such as to facilitate falling asleep, staying asleep, and/or waking up. Respiration information (e.g., respiration rate, time between breaths, maximal inspiration information, maximal expiration information, respiration rate variability, respiration morphology information, and the like) can be extracted from the biometric sensor data and used to establish a target respiration pattern. An entrainment signal can be determined from the target respiration pattern and then used to present an entrainment stimulus (e.g., via audio, visual, tactile, or other stimuli) to the user.

### CROSS-REFERENCE TO RELATED APPLICATIONS

This application claims the benefit of, and priority to, U.S. Provisional Patent Application No. 63/238,410 filed on Aug. 30, 2021, which is hereby incorporated by reference herein in its entirety.

### TECHNICAL FIELD

The present disclosure relates generally to systems and methods for facilitating sleep, and more particularly, to systems and methods for encouraging hyper-personalized breathing patterns before, during, or after sleep.

### BACKGROUND

Many individuals suffer from sleep-related and/or respiratory-related disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, insomnia, and chest wall disorders. These disorders are often treated using a respiratory therapy system.

Specifically, many individuals suffer from difficulty falling asleep. Further, as individuals experience recurring periods of bad sleep or difficulty falling asleep, those individuals may begin to consciously or subconsciously associate going to sleep with having a bad sleep. For some individuals, periods prior to and immediately prior to going to bed may be marked by sympathetic autonomic nervous system activation, whether from physical or mental activity, which can make falling asleep difficult.

Difficulty falling asleep can be a sleep-related disorder itself, but can also affect other sleep-related disorders. While certain disorders can be effectively treated using a respiratory therapy system, use of such a respiratory therapy system will not be fully effective until the individual's trouble falling asleep is managed.

Certain paced breathing programs exist, but due to their nature, are not personalized for each user. Some paced breathing programs exist to identify and help individuals achieve optimized breathing patterns for athletic performance, such as WO 2016/074042, but such programs do not assist an individual in various important aspects, such as falling asleep, staying asleep, or otherwise engaging in a sleep session. The present disclosure is directed to solving these and other problems.

### SUMMARY

According to some implementations of the present disclosure, a method includes receiving biometric sensor data associated with a user. The method further includes extracting respiration information from the biometric sensor data. The method further includes determining a target respiration pattern. The method further includes presenting an entrainment program to the user based at least in part on the target respiration pattern. Presenting the entrainment program facilitates entraining a respiration pattern of the user towards the target respiration pattern. Presenting the entrainment program includes determining an entrainment signal based at least in part on the respiration information and the target respiration pattern. Presenting the entrainment program further includes presenting an entrainment stimulus to the user based at least in part on the entrainment signal.

According to some implementations of the present disclosure, a system includes an electronic interface, a memory, and a control system. The electronic interface is configured to receive biometric sensor data associated with a user. The memory stores machine-readable instructions. The control system includes one or more processors configured to execute the machine-readable instructions to extract respiration information from the biometric sensor data. The control system is further configured to determine a target respiration pattern. The control system is further configured to present an entrainment program to the user based at least in part on the target respiration pattern. Presenting the entrainment program facilitates entraining a respiration pattern of the user towards the target respiration pattern. Presenting the entrainment program includes determining an entrainment signal based at least in part on the respiration information and the target respiration pattern. Presenting the entrainment program further includes presenting an entrainment stimulus to the user based at least in part on the entrainment signal.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2 is a perspective view of at least a portion of the system of FIG. 1, a user, and a bed partner, according to some implementations of the present disclosure.

FIG. 3 illustrates an exemplary timeline for a sleep session, according to some implementations of the present disclosure.

FIG. 4 illustrates an exemplary hypnogram associated with the sleep session of FIG. 3, according to some implementations of the present disclosure.

FIG. 5 is a flowchart depicting a process for presenting an entrainment program according to some implementations of the present disclosure.

FIG. 6 is a flowchart depicting a process for using an entrainment program according to some implementations of the present disclosure.

FIG. 7 is a flowchart depicting a process for presenting an entrainment program based on stress level according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

As disclosed in further detail herein, an intelligent entrainment program can make use of received biometric sensor data to provide hyper-personalized guidance to entrain a user's respiration pattern towards a target respiration pattern. The entrainment program can be used for sleep-related therapy, such as to facilitate falling asleep, staying asleep, and/or waking up. Respiration information (e.g., respiration rate, time between breaths, maximal inspiration information, maximal expiration information, respiration rate variability, respiration morphology information, and the like) can be extracted from the biometric sensor data and used to establish a target respiration pattern. An entrainment signal can be determined from the target respiration pattern and then used to present an entrainment stimulus (e.g., via audio, visual, tactile, or other stimuli) to the user. Certain aspects and features of the present disclosure assist a user in engaging in a sleep session, such as facilitating falling asleep, facilitating staying asleep, facilitating achieving better quality sleep, facilitating achieving more time spent in specific sleep states and/or sleep stages, facilitating waking up, and/or facilitating achieving a greater feeling of restfulness after waking up.

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, insomnia, and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Many individuals suffer from insomnia, a condition which is generally characterized by a dissatisfaction with sleep quality or duration (e.g., difficulty initiating sleep, frequent or prolonged awakenings after initially falling asleep, and an early awakening with an inability to return to sleep). It is estimated that over 2.6 billion people worldwide experience some form of insomnia, and over 750 million people worldwide suffer from a diagnosed insomnia disorder. In the United States, insomnia causes an estimated gross economic burden of $107.5 billion per year, and accounts for 13.6% of all days out of role and 4.6% of injuries requiring medical attention. Recent research also shows that insomnia is the second most prevalent mental disorder, and that insomnia is a primary risk factor for depression.

Nocturnal insomnia symptoms generally include, for example, reduced sleep quality, reduced sleep duration, sleep-onset insomnia, sleep-maintenance insomnia, late insomnia, mixed insomnia, and/or paradoxical insomnia. Sleep-onset insomnia is characterized by difficulty initiating sleep at bedtime. Sleep-maintenance insomnia is characterized by frequent and/or prolonged awakenings during the night after initially falling asleep. Late insomnia is characterized by an early morning awakening (e.g., prior to a target or desired wakeup time) with the inability to go back to sleep. Comorbid insomnia refers to a type of insomnia where the insomnia symptoms are caused at least in part by a symptom or complication of another physical or mental condition (e.g., anxiety, depression, medical conditions, and/or medication usage). Mixed insomnia refers to a combination of attributes of other types of insomnia (e.g., a combination of sleep-onset, sleep-maintenance, and late insomnia symptoms). Paradoxical insomnia refers to a disconnect or disparity between the user's perceived sleep quality and the user's actual sleep quality.

Diurnal (e.g., daytime) insomnia symptoms include, for example, fatigue, reduced energy, impaired cognition (e.g., attention, concentration, and/or memory), difficulty functioning in academic or occupational settings, and/or mood disturbances. These symptoms can lead to psychological complications such as, for example, lower mental (and/or physical) performance, decreased reaction time, increased risk of depression, and/or increased risk of anxiety disorders. Insomnia symptoms can also lead to physiological complications such as, for example, poor immune system function, high blood pressure, increased risk of heart disease, increased risk of diabetes, weight gain, and/or obesity.

Co-morbid Insomnia and Sleep Apnea (COMISA) refers to a type of insomnia where the subject experiences both insomnia and obstructive sleep apnea (OSA). OSA can be measured based on an Apnea-Hypopnea Index (AHI) and/or oxygen desaturation levels. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild OSA. An AHI that is greater than or equal to 15, but less than 30 is considered indicative of moderate OSA. An AHI that is greater than or equal to 30 is considered indicative of severe OSA. In children, an AHI that is greater than 1 is considered abnormal.

Insomnia can also be categorized based on its duration. For example, insomnia symptoms are considered acute or transient if they occur for less than 3 months. Conversely, insomnia symptoms are considered chronic or persistent if they occur for 3 months or more, for example. Persistent/chronic insomnia symptoms often require a different treatment path than acute/transient insomnia symptoms.

Known risk factors for insomnia include gender (e.g., insomnia is more common in females than males), family history, and stress exposure (e.g., severe and chronic life events). Age is a potential risk factor for insomnia. For example, sleep-onset insomnia is more common in young adults, while sleep-maintenance insomnia is more common in middle-aged and older adults. Other potential risk factors for insomnia include race, geography (e.g., living in geographic areas with longer winters), altitude, and/or other sociodemographic factors (e.g. socioeconomic status, employment, educational attainment, self-rated health, etc.).

Mechanisms of insomnia include predisposing factors, precipitating factors, and perpetuating factors. Predisposing factors include hyperarousal, which is characterized by increased physiological arousal during sleep and wakefulness. Measures of hyperarousal include, for example, increased levels of cortisol, increased activity of the autonomic nervous system (e.g., as indicated by increase resting heart rate and/or altered heart rate), increased brain activity (e.g., increased EEG frequencies during sleep and/or increased number of arousals during REM sleep), increased metabolic rate, increased body temperature and/or increased activity in the pituitary-adrenal axis. Precipitating factors include stressful life events (e.g., related to employment or education, relationships, etc.) Perpetuating factors include excessive worrying about sleep loss and the resulting consequences, which may maintain insomnia symptoms even after the precipitating factor has been removed.

Conventionally, diagnosing or screening insomnia (including identifying a type or insomnia and/or specific symptoms) involves a series of steps. Often, the screening process begins with a subjective complaint from a patient (e.g., they cannot fall or stay sleep).

Next, the clinician evaluates the subjective complaint using a checklist including insomnia symptoms, factors that influence insomnia symptoms, health factors, and social factors. Insomnia symptoms can include, for example, age of onset, precipitating event(s), onset time, current symptoms (e.g., sleep-onset, sleep-maintenance, late insomnia), frequency of symptoms (e.g., every night, episodic, specific nights, situation specific, or seasonal variation), course since onset of symptoms (e.g., change in severity and/or relative emergence of symptoms), and/or perceived daytime consequences. Factors that influence insomnia symptoms include, for example, past and current treatments (including their efficacy), factors that improve or ameliorate symptoms, factors that exacerbate insomnia (e.g., stress or schedule changes), factors that maintain insomnia including behavioral factors (e.g., going to bed too early, getting extra sleep on weekends, drinking alcohol, etc.) and cognitive factors (e.g., unhelpful beliefs about sleep, worry about consequences of insomnia, fear of poor sleep, etc.). Health factors include medical disorders and symptoms, conditions that interfere with sleep (e.g., pain, discomfort, treatments), and pharmacological considerations (e.g., alerting and sedating effects of medications). Social factors include work schedules that are incompatible with sleep, arriving home late without time to wind down, family and social responsibilities at night (e.g., taking care of children or elderly), stressful life events (e.g., past stressful events may be precipitants and current stressful events may be perpetuators), and/or sleeping with pets.

After the clinician completes the checklist and evaluates the insomnia symptoms, factors that influence the symptoms, health factors, and/or social factors, the patient is often directed to create a daily sleep diary and/or fill out a questionnaire (e.g., Insomnia Severity Index or Pittsburgh Sleep Quality Index). Thus, this conventional approach to insomnia screening and diagnosis is susceptible to error(s) because it relies on subjective complaints rather than objective sleep assessment. There may be a disconnect between patient's subjective complaint(s) and the actual sleep due to sleep state misperception (paradoxical insomnia).

In addition, the conventional approach to insomnia diagnosis does not rule out other sleep-related disorders such as, for example, Periodic Limb Movement Disorder (PLMD), Restless Leg Syndrome (RLS), Sleep-Disordered Breathing (SDB), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping. While these other sleep-related disorders may have similar symptoms as insomnia, distinguishing these other sleep-related disorders from insomnia is useful for tailoring an effective treatment plan distinguishing characteristics that may call for different treatments. For example, fatigue is generally a feature of insomnia, whereas excessive daytime sleepiness is a characteristic feature of other disorders (e.g., PLMD) and reflects a physiological propensity to fall asleep unintentionally.

Once diagnosed, insomnia can be managed or treated using a variety of techniques or providing recommendations to the patient. Generally, the patient can be encouraged or recommended to generally practice healthy sleep habits (e.g., plenty of exercise and daytime activity, have a routine, no bed during the day, eat dinner early, relax before bedtime, avoid caffeine in the afternoon, avoid alcohol, make bedroom comfortable, remove bedroom distractions, get out of bed if not sleepy, try to wake up at the same time each day regardless of bed time) or discouraged from certain habits (e.g., do not work in bed, do not go to bed too early, do not go to bed if not tired). The patient can additionally or alternatively be treated using sleep medicine and medical therapy such as prescription sleep aids, over-the-counter sleep aids, and/or at-home herbal remedies.

The patient can also be treated using cognitive behavior therapy (CBT) or cognitive behavior therapy for insomnia (CBT-I), which generally includes sleep hygiene education, relaxation therapy, stimulus control, sleep restriction, and sleep management tools and devices. Sleep restriction is a method designed to limit time in bed (the sleep window or duration) to actual sleep, strengthening the homeostatic sleep drive. The sleep window can be gradually increased over a period of days or weeks until the patient achieves an optimal sleep duration. Stimulus control includes providing the patient a set of instructions designed to reinforce the association between the bed and bedroom with sleep and to reestablish a consistent sleep-wake schedule (e.g., go to bed only when sleepy, get out of bed when unable to sleep, use the bed for sleep only (e.g., no reading or watching TV), wake up at the same time each morning, no napping, etc.) Relaxation training includes clinical procedures aimed at reducing autonomic arousal, muscle tension, and intrusive thoughts that interfere with sleep (e.g., using progressive muscle relaxation). Cognitive therapy is a psychological approach designed to reduce excessive worrying about sleep and reframe unhelpful beliefs about insomnia and its daytime consequences (e.g., using Socratic question, behavioral experiences, and paradoxical intention techniques). Sleep hygiene education includes general guidelines about health practices (e.g., diet, exercise, substance use) and environmental factors (e.g., light, noise, excessive temperature) that may interfere with sleep. Mindfulness-based interventions can include, for example, meditation.

In some cases, insomnia or insomnia-related parameters can be identified, such as described in WO 2021/084478A1. In some cases, hyperarousal can be identified and/or measured. Hyperarousal is characterized by increased physiological activity and can be indicative of a stress level of the user. In some cases, hyperarousal level of a user can be determined based on a sleep-wake signal, received physiological information, and/or other data (e.g., personal data). For example, the hyperarousal level can be determined by comparing a self-reported subjective stress level of the user included in personal data to previously recorded subjective stress levels for the user and/or a population norm. In another example, the hyperarousal level can be determined based on breathing of the user during the sleep session (e.g., breathing rate, breath variability, breath duration, breath interval, average breathing rate, breathing during each sleep stage). For another example, the hyperarousal level can be determined based on movement of the user during the sleep session (e.g., based on data from a motion sensor). In a further example, the hyperarousal level can be determined based heart rate data for the user during the sleep session or during the daytime.

The gold standard for diagnosing sleep apnea is polysomnography (PSG). PSG utilizes multiple sensing channels including electroencephalography (EEG), electrooculography (EOG), electromyography (EMG), electrocardiography (ECG), and pulse oximetry, as well as airflow and respiratory effort to assess underlying causes of sleep disturbances. However, due to the inconvenience of performing an in-lab PSG and in part due to the global COVID pandemic, Home Sleep Apnea Testing (HSAT) based on peripheral arterial tonometry (PAT) have rapidly gained popularity and currently comprise the most widely deployed category of HSAT. PAT-based HSATs obtain most of its sensing modalities from finger photoplethysmography (PPG), from which it derives the blood oxygen saturation (SpO2), pulse rate (PR), and PAT. PAT-based HSATs allows for minimally invasive multi-night testing and are available in a fully disposable format. An example of such a system is called NightOwl™, which was described by Massie et al. (“An evaluation of the Night Owl home sleep apnea testing system,” Journal of Clinical Sleep Medicine, vol. 14, no. 10, pp. 1791-1796, October 2018, doi: 10.5664/jcsm.7398). It includes a finger probe of the size of a fingertip that senses peripheral arterial tone, together with actigraphy and oximetry, and works with cloud-based analysis software. The analysis determines respiratory-related information, including occurrence of respiratory events (such as obstructive and central apnea events). The device and analyses are described in US2020/0015737A1, WO2021260190A1, and WO2021260192A1, each of which is incorporated herein in its entirety.

As disclosed herein, not only is such a device suitable for diagnosing sleep apnea, the device may be used to determine, or derive from the peripheral arterial tone signal determined from the PPG signal, for example, a respiration rate, heart rate, heart rate variability, limb and/or body motion, and from which a user's stress level may be inferred. Furthermore, the peripheral arterial tone signal rises and falls with changes in the sympathetic nervous system and thus may be used to monitor sympathetic nervous system activity as an indicator of user stress levels. As such, these devices may be used to monitor stress levels and assess the effect of entrainment stimuli on stress levels.

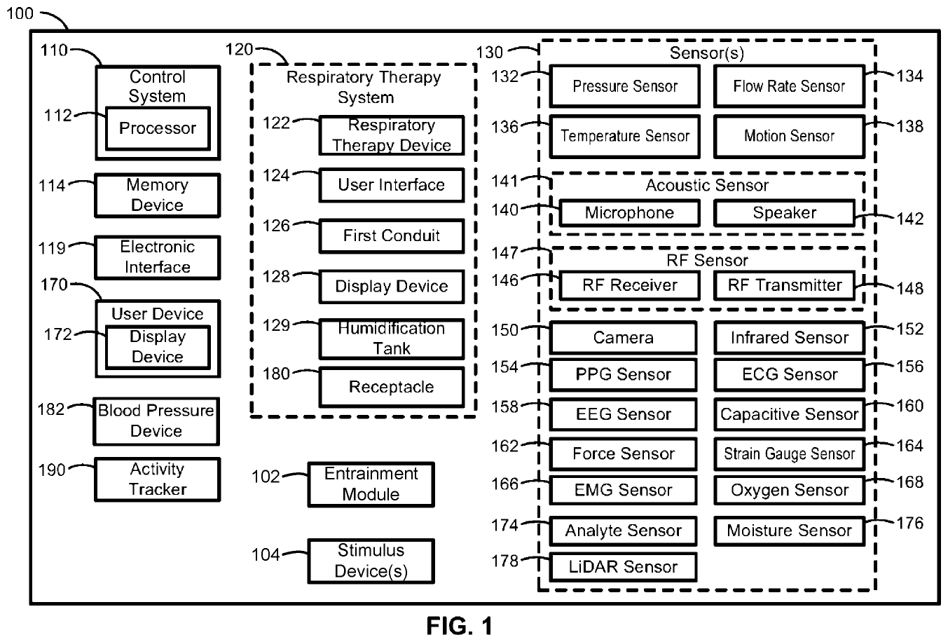

Referring to FIG. 1, a functional block diagram is illustrated, of a system 100 for presenting an entrainment program to a user, such as a user of a respiratory therapy system. The system 100 includes a entrainment module 102, a control system 110, a memory device 114, an electronic interface 119, one or more sensors 130, and one or more user devices 170. In some implementations, the system 100 further optionally includes a respiratory therapy system 120, a blood pressure device 182, an activity tracker 190, or any combination thereof.

The entrainment module 102 determines and/or facilitates presentation of an entrainment program based at least in part on biometric sensor data (e.g., biometric sensor data acquired from the one or more sensors 130, as disclosed in further detail herein). Some or all of the entrainment module 102 can be implemented by and/or make use of any other elements of system 100.

The entrainment module 102 can generate an entrainment signal from the biometric sensor data. The entrainment signal can include information indicative of a rhythm, a morphology, a rate, and/or other features of a desired respiration pattern. For example, an entrainment signal can be a sine wave at 0.333 Hz, which can be indicative of a respiration rate of at or approximately 20 breaths per minute (bpm). In another example, an entrainment signal can be a non-sinusoidal wave that changes frequency over time, which can be indicative of a respiration morphology (e.g., timing and extent of inhalation and exhalation over time) and a changing respiration rate.

The entrainment signal can be used to present an entrainment stimulus to the user via one or more stimulus devices 104. Any suitable device that can present discernable input to the user can be used as a stimulus device 104. In some cases, the one or more stimulus devices 104 can include (i) a tactile stimulus device (e.g., a vibrating motor); (ii) a visual stimulus device (e.g., a display device, such as display device 172); (iii) an audio stimulus device (e.g., a speaker, such as speaker 142); (iv) an airflow stimulus device (e.g., a respiratory therapy device, such as respiratory therapy device 122); or (v) any combination of i-iv. The entrainment signal can be used to present a single entrainment stimulus (e.g., a sound of lapping ocean waves) or multiple entrainment stimuli (e.g., a sound of lapping ocean waves and a visual cue of an expanding and contracting circle).

The control system 110 includes one or more processors 112 (hereinafter, processor 112). The control system 110 is generally used to control (e.g., actuate) the various components of the system 100 (e.g., including stimulus device(s) 104) and/or analyze data obtained and/or generated by the components of the system 100 (e.g., entrainment module 102). The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, the activity tracker 190, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, the activity tracker 190, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more sleep sessions), entrainment parameters associated with the user, or any combination thereof. The demographic information can include, for example, information indicative of an age of the user, a gender of the user, a race of the user, an ethnicity of the user, a geographic location of the user, a travel history of the user, a relationship status, a status of whether the user has one or more pets, a status of whether the user has a family, a family history of health conditions, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The medical information data can include results from one or more of a polysomnography (PSG) test, a CPAP titration, or a home sleep test (HST), respiratory therapy system settings from one or more sleep sessions, sleep related respiratory events from one or more sleep sessions, or any combination thereof. The self-reported user feedback can include information indicative of a self-reported subjective therapy score (e.g., poor, average, excellent), a self-reported subjective stress level of the user, a self-reported subjective fatigue level of the user, a self-reported subjective health status of the user, a recent life event experienced by the user, or any combination thereof. The entrainment parameters can include various information associated with one or more entrainment programs, such as information regarding the user's historical entrainment programs, the effects of one or more historical entrainment programs, and the like. The user profile information can be updated at any time, such as daily (e.g. between sleep sessions), weekly, monthly or yearly. In some implementations, the memory device 114 stores media content that can be displayed on the display device 128 and/or the display device 172.

The electronic interface 119 is configured to receive data (e.g., physiological data, flow rate data, pressure data, motion data, acoustic data, etc.) from the one or more sensors 130 such that the data can be stored in the memory device 114 and/or analyzed by the processor 112 of the control system 110. The received data, such as physiological data, flow rate data, pressure data, motion data, acoustic data, etc., may be used to determine and/or calculate physiological parameters. The electronic interface 119 can communicate with the one or more sensors 130 using a wired connection or a wireless connection (e.g., using an RF communication protocol, a Wi-Fi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The respiratory therapy system 120 can include a respiratory pressure therapy (RPT) device 122 (referred to herein as respiratory device 122), a user interface 124, a conduit 126 (also referred to as a tube or an air circuit), a display device 128, a humidification tank 129, a receptacle 180 or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver pressurized air at a pressure of at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory device 122, the user interface 124, and the conduit 126 form an air pathway fluidly coupled with an airway of the user. The pressurized air also increases the user's oxygen intake during sleep.

Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cmH₂O relative to ambient pressure. For other forms of therapy, such as the delivery of oxygen, the user interface may not include a seal sufficient to facilitate delivery to the airways of a supply of gas at a positive pressure of about 10 cmH₂O.

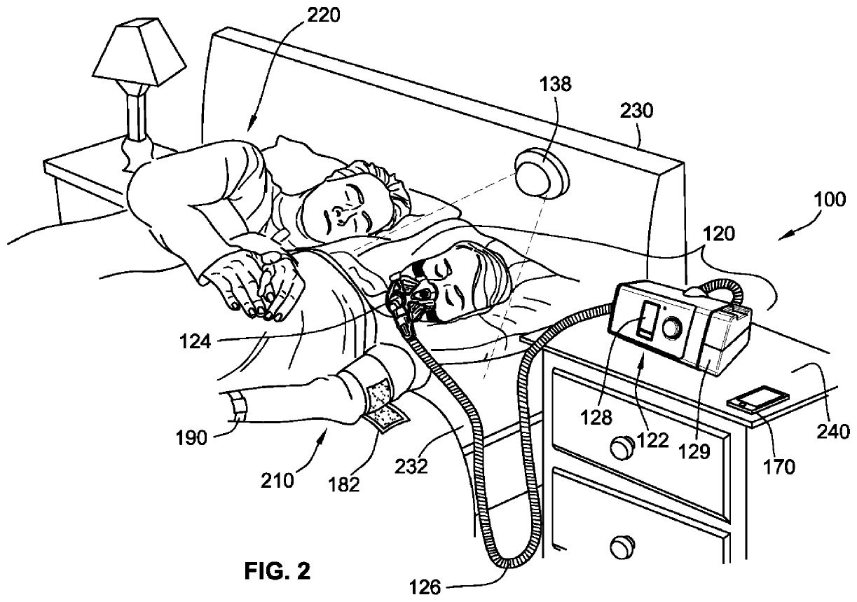

As shown in FIG. 2, in some implementations, the user interface 124 is or includes a facial mask (e.g., a full face mask) that covers the nose and mouth of the user. Alternatively, in some implementations, the user interface 124 is a nasal mask that provides air to the nose of the user or a nasal pillow mask that delivers air directly to the nostrils of the user. The user interface 124 can include a plurality of straps (e.g., including hook and loop fasteners) for positioning and/or stabilizing the interface on a portion of the user (e.g., the face) and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 (also referred to as an air circuit or tube) allows the flow of air between two components of the respiratory therapy system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, a humidity sensor, a temperature sensor, or more generally any of the other sensors 130 described herein). These one or more sensors can be used, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score and/or a therapy score (such as a myAir™ score, such as described in WO 2016/061629, which is hereby incorporated by reference herein in its entirety), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122. The humidification tank 129 includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory device 122 or the conduit 126 can include a waterless humidifier. The waterless humidifier can incorporate sensors that interface with other sensor positioned elsewhere in system 100.

In some implementations, the system 100 can be used to deliver at least a portion of a substance from a receptacle 180 to the air pathway the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) any combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. A motion sensor 138, a blood pressure device 182, and an activity tracker 190 are shown, although any one or more sensors 130 can be used to generate or monitor physiological parameters during a therapy, sleeping, and/or resting session of the user 210.

The user interface 124 is a facial mask (e.g., a full face mask) that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps (e.g., including hook and loop fasteners) for positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents for permitting the escape of carbon dioxide and other gases exhaled by the user 210. In other implementations, the user interface 124 is a mouthpiece (e.g., a night guard mouthpiece molded to conform to the user's teeth, a mandibular repositioning device, etc.) for directing pressurized air into the mouth of the user 210.

The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Generally, a user who is prescribed usage of the respiratory therapy system 120 will tend to experience higher quality sleep and less fatigue during the day after using the respiratory therapy system 120 during the sleep compared to not using the respiratory therapy system 120 (especially when the user suffers from sleep apnea or other sleep related disorders). For example, the user 210 may suffer from obstructive sleep apnea and rely on the user interface 124 (e.g., a full face mask) to deliver pressurized air from the respiratory device 122 via conduit 126. The respiratory device 122 can be a continuous positive airway pressure (CPAP) machine used to increase air pressure in the throat of the user 210 to prevent the airway from closing and/or narrowing during sleep. For someone with sleep apnea, their airway can narrow or collapse during sleep, reducing oxygen intake, and forcing them to wake up and/or otherwise disrupt their sleep. The CPAP machine prevents the airway from narrowing or collapsing, thus minimizing the occurrences where she wakes up or is otherwise disturbed due to reduction in oxygen intake. While the respiratory device 122 strives to maintain a medically prescribed air pressure or pressures during sleep, the user can experience sleep discomfort due to the therapy.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a Light Detection and Ranging (LiDAR) sensor 178, an electrodermal sensor, an accelerometer, an electrooculography (EOG) sensor, a light sensor, a humidity sensor, an air quality sensor, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the Light Detection and Ranging (LiDAR) sensor 178 more generally, the one or more sensors 130 can include any combination and any number of each of the sensors described and/or shown herein.

As described herein, the system 100 generally can be used to generate data (e.g., physiological data, flow rate data, pressure data, motion data, acoustic data, etc.) associated with a user (e.g., a user of the respiratory therapy system 120 shown in FIG. 2) before, during, and/or after a sleep session. The generated data can be analyzed to generate one or more physiological parameters (e.g., before, during, and/or after a sleep session) and/or sleep-related parameters (e.g., during a sleep session), which can include any parameter, measurement, etc. related to the user. Examples of the one or more physiological parameters include a respiration pattern, a respiration rate, an inspiration amplitude, an expiration amplitude, a heart rate, heart rate variability, a length of time between breaths, a time of maximal inspiration, a time of maximal expiration, a forced breath parameter (e.g., distinguishing releasing breath from forced exhalation), respiration variability, breath morphology (e.g., the shape of one or more breaths), movement of the user 210, temperature, EEG activity, EMG activity, ECG data, a sympathetic response parameter, a parasympathetic response parameter, and the like. The one or more sleep-related parameters that can be determined for the user 210 during the sleep session include, for example, an Apnea-Hypopnea Index (AHI) score, a sleep score, a therapy score, a flow signal, a pressure signal, a respiration signal, a respiration pattern, a respiration rate, an inspiration amplitude, an expiration amplitude, an inspiration-expiration ratio, a number of events (e.g., apnea events) per hour, a pattern of events, a sleep state and/or sleep stage, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, flow rate data, pressure data, motion data, acoustic data, etc. In some implementations, the data generated by one or more of the sensors 130 can be used by the control system 110 to determine the duration of sleep and sleep quality of user 210. For example, a sleep-wake signal associated with the user 210 during the sleep session and one or more sleep-related parameters. The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more of the sensors, such as sensors 130, are described in, for example, WO 2014/047310, US 2014/0088373, WO 2017/132726, WO 2019/122413, and WO 2019/122414, each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory device 122, or any combination thereof during the sleep session.

The event(s) can include snoring, apneas, central apneas, obstructive apneas, mixed apneas, hypopneas, a mouth leak, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, or any combination thereof. In some implementations, mouth leak can include continuous mouth leak, or valve-like mouth leak (i.e. varying over the breath duration) where the lips of a user, typically using a nasal/nasal pillows mask, pop open on expiration. Mouth leak can lead to dryness of the mouth, bad breath, and is sometimes colloquially referred to as “sandpaper mouth.”

The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof.

The data generated by the one or more sensors 130 (e.g., physiological data, flow rate data, pressure data, motion data, acoustic data, etc.) can also be used to determine a respiration signal. The respiration signal is generally indicative of respiration or breathing of the user. The respiration signal can be indicative of a respiration pattern, which can include, for example, a respiration rate, a respiration rate variability, an inspiration amplitude, an expiration amplitude, an inspiration-expiration ratio, and other respiration-related parameters, as well as any combination thereof. In some cases, during a sleep session, the respiration signal can include a number of events per hour (e.g., during sleep), a pattern of events, pressure settings of the respiratory device 122, or any combination thereof. The event(s) can include snoring, apneas (e.g., central apneas, obstructive apneas, mixed apneas, and hypopneas), a mouth leak, a mask leak (e.g., from the user interface 124), a restless leg, a sleeping disorder, choking, an increased heart rate, labored breathing, an asthma attack, an epileptic episode, a seizure, or any combination thereof.

Generally, the sleep session includes any point in time after the user 210 has laid or sat down in the bed 230 (or another area or object on which they intend to sleep), and/or has turned on the respiratory device 122 and/or donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory device 122, and/or gets out of bed 230. In some implementations, the sleep session can include additional periods of time, or can be limited to only some of the above-disclosed time periods. For example, the sleep session can be defined to encompass a period of time beginning when the respiratory device 122 begins supplying the pressurized air to the airway or the user 210, ending when the respiratory device 122 stops supplying the pressurized air to the airway of the user 210, and including some or all of the time points in between, when the user 210 is asleep or awake.

In some cases, a pre-asleep period can be defined as a period of time before a user falls asleep (e.g., before the user enters light sleep, deep sleep, REM sleep), which can include time before and/or after the user has laid or sat down in the bed 230 (or another area or object on which they intend to sleep). In some cases, the personalized entrainment as disclosed herein can be used during this pre-sleep period, although that need not always be the case. In some cases, for example, personalized entrainment can be used during a sleep session (e.g., while the user is asleep or while the user is periodically awake between light sleep, deep sleep, or REM sleep) and/or after a sleep session (e.g., after the user has awoken and decides to stay awake). In some cases, the personalized entrainment as disclosed herein can be used during the pre-sleep period, continue during the sleep session (e.g., in the same or modified form) and/or after the sleep session has ended (e.g., in the same or modified form).

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory device 122. the user interface 124, or the conduit 126. The pressure sensor 132 can be used to determine an air pressure in the respiratory device 122, an air pressure in the conduit 126, an air pressure in the user interface 124, or any combination thereof. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The flow rate sensor 134 can be used to generate flow rate data associated with the user 210 (FIG. 2) of the respiratory device 122 during the sleep session. Examples of flow rate sensors (such as, for example, the flow rate sensor 134) are described in WO 2012/012835, which is hereby incorporated by reference herein in its entirety. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user.

The temperature sensor 136 outputs temperature data that can be stored in the memory device 114 and/or analyzed by the processor 112 of the control system 110. In some implementations, the temperature sensor 136 generates temperature data indicative of a core body temperature of the user 210 (FIG. 2), a skin temperature of the user 210, a temperature of the air flowing from the respiratory device 122 and/or through the conduit 126, a temperature of the air in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state or sleep stage of the user; for example, via a respiratory movement of the user. In some implementations, the motion data from the motion sensor 138 can be used in conjunction with additional data from another sensor 130 to determine the sleep state or sleep stage of the user. In some implementations, the motion data can be used to determine a location, a body position, and/or a change in body position of the user.

The microphone 140 outputs sound data that can be stored in the memory device 114 and/or analyzed by the processor 112 of the control system 110. The microphone 140 can be used to record sound(s) during a sleep session (e.g., sounds from the user 210) to determine (e.g., using the control system 110) one or more sleep related parameters, which may include one or more events (e.g., respiratory events), as described in further detail herein. The microphone 140 can be coupled to or integrated in the respiratory device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves. In one or more implementations, the sound waves can be audible to a user of the system 100 (e.g., the user 210 of FIG. 2) or inaudible to the user of the system (e.g., ultrasonic sound waves). The speaker 142 can be used, for example, as an alarm clock or to play an alert or message to the user 210 (e.g., in response to an identified body position and/or a change in body position). In some implementations, the speaker 142 can be used to communicate the audio data generated by the microphone 140 to the user. The speaker 142 can be coupled to or integrated in the respiratory device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g. a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters (e.g., an identified body position and/or a change in body position) and/or respiration-related parameters described in herein such as, for example, a respiration pattern, a respiration signal (from which, e.g., breath morphology may be determined), a respiration rate, an inspiration amplitude, an expiration amplitude, an inspiration-expiration ratio, a number of events per hour, a pattern of events, a sleep state, a sleep stage, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some cases, a microphone 140 and/or speaker 142 can be incorporated into a separate device, such as body-worn device, such as one or a set of earphones or headphones. In some cases, such a device can include other of the one or more sensors 130.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location and/or a body position of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication could be Wi-Fi, Bluetooth, or etc.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location and/or a body position of the user, to determine chest movement of the user 210, to determine air flow of the mouth and/or nose of the user 210, to determine a time when the user 210 enters the bed 230, and to determine a time when the user 210 exits the bed 230. The camera 150 can also be used to track eye movements, pupil dilation (if one or both of the user 210's eyes are open), blink rate, or any changes during REM sleep.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc. In some cases, the PPG sensor 154 can be a non-contact PPG sensor capable of PPG at a distance. In some cases, a PPG sensor 154 can be used in the determination of a pulse arrival time (PAT). PAT can be a determination of the time interval needed for a pulse wave to travel from the heart to a distal location on the body, such as a finger or other location. In other words, the PAT can be determined by measuring the time interval between the R wave of an ECG and a peak of the PPG. In some cases, baseline changes in the PPG signal can be used to derive a respiratory signal, and thus respiratory information, such as respiratory rate. In some cases, the PPG signal can provide SpO2 data, which can be used in the detection of sleep-related disorders, such as OSA.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein. In some cases, the amplitude and/or morphology changes in the ECG electrical trace can be used to identify a breathing curve, and thus respiratory information, such as a respiratory rate.

In some cases, an ECG signal and/or a PPG signal can be used in concert with a secondary estimate of parasympathetic and/or sympathetic innervation, such as via a galvanic skin response (GSR) sensor. Such signals can be used to identify what actual breathing curve is occurring, and if it has a positive, neutral, or negative impact on the stress level of the individual.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state or sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a GSR sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near the user 210's mouth to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the user 210's nose to detect analytes in breath exhaled through the user's nose. In still other implementations, the analyte sensor 174 can be positioned near the user 210's mouth when the user interface 124 is a nasal mask or a nasal pillow mask. In some implementations, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the user 210's mouth or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be positioned in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example, the air inside the user 210's bedroom. The moisture sensor 176 can also be used to track the user 210's biometric response to environmental changes.

One or more Light Detection and Ranging (LiDAR) sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor(s) 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, an orientation sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170, the entrainment module 102, the stimulus device(s) 104, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not physically and/or communicatively coupled to the respiratory device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more physiological parameters, which can include a respiration signal, a respiration rate, a respiration pattern or morphology, respiration rate variability, an inspiration amplitude, an expiration amplitude, an inspiration-expiration ratio, a length of time between breaths, a time of maximal inspiration, a time of maximal expiration, a forced breath parameter (e.g., distinguishing releasing breath from forced exhalation), an occurrence of one or more events, a number of events per hour, a pattern of events, a sleep state, a sleep stage, an apnea-hypopnea index (AHI), a heart rate, heart rate variability, movement of the user 210, temperature, EEG activity, EMG activity, ECG data, a sympathetic response parameter, a parasympathetic response parameter or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased blood pressure, or any combination thereof. Many of these physiological parameters are sleep-related parameters, although in some cases the data from the one or more sensors 130 can be analyzed to determine one or more non-physiological parameters, such as non-physiological sleep-related parameters. Non-physiological parameters can also include operational parameters of the respiratory therapy system, including flow rate, pressure, humidity of the pressurized air, speed of motor, etc. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s), optionally with a display, such as Google Home™, Google Nest™, Amazon Echo™, Amazon Echo Show™, Alexa™-enabled devices, etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 182 is generally used to aid in generating physiological data for determining one or more blood pressure measurements associated with a user. The blood pressure device 182 can include at least one of the one or more sensors 130 to measure, for example, a systolic blood pressure component and/or a diastolic blood pressure component.

In some implementations, the blood pressure device 182 is a sphygmomanometer including an inflatable cuff that can be worn by a user and a pressure sensor (e.g., the pressure sensor 132 described herein). For example, as shown in the example of FIG. 2, the blood pressure device 182 can be worn on an upper arm of the user 210. In such implementations where the blood pressure device 182 is a sphygmomanometer, the blood pressure device 182 also includes a pump (e.g., a manually operated bulb) for inflating the cuff. In some implementations, the blood pressure device 182 is coupled to the respiratory device 122 of the respiratory therapy system 120, which in turn delivers pressurized air to inflate the cuff. More generally, the blood pressure device 182 can be communicatively coupled with, and/or physically integrated in (e.g., within a housing), the control system 110, the memory 114, the respiratory therapy system 120, the user device 170, and/or the activity tracker 190.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation level (SpO₂), electrodermal activity (also known as skin conductance or galvanic skin response), a position of the user, a posture of the user, or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user. Alternatively still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory 114, the respiratory therapy system 120, and/or the user device 170, and/or the blood pressure device 182.

While the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for analyzing data associated with a user's use of the respiratory therapy system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, the user device 170, and the blood pressure device 182 and/or activity tracker 190. As yet another example, a third alternative system includes the control system 110, the memory device 114, the respiratory therapy system 120, at least one of the one or more sensors 130, and the user device 170. As a further example, a fourth alternative system includes the control system 110, the memory device 114, the respiratory therapy system 120, at least one of the one or more sensors 130, the user device 170, and the blood pressure device 182 and/or activity tracker 190. Thus, various systems can be formed using any portion or portions of the components shown and described herein and/or in combination with one or more other components.

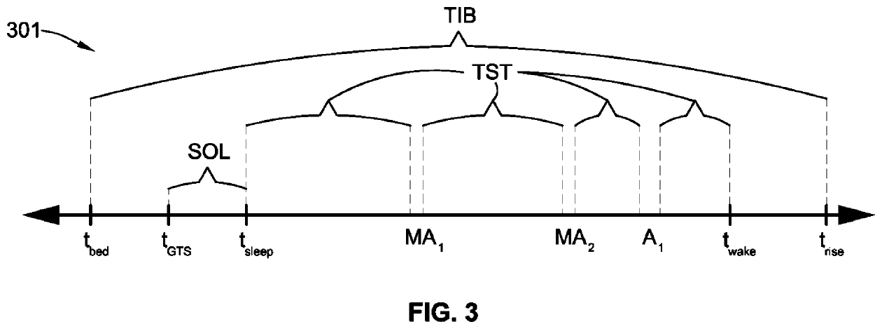

Referring to the timeline 301 in FIG. 3, the enter bed time t_{bed} is associated with the time that the user initially enters the bed (e.g., bed 230 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 4 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 3 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (tGTS) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 4 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (tGTS) and ending at the wake-up time (t_{wake}). In some implementations, a sleep session is defined as starting at the go-to-sleep time (tGTS) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

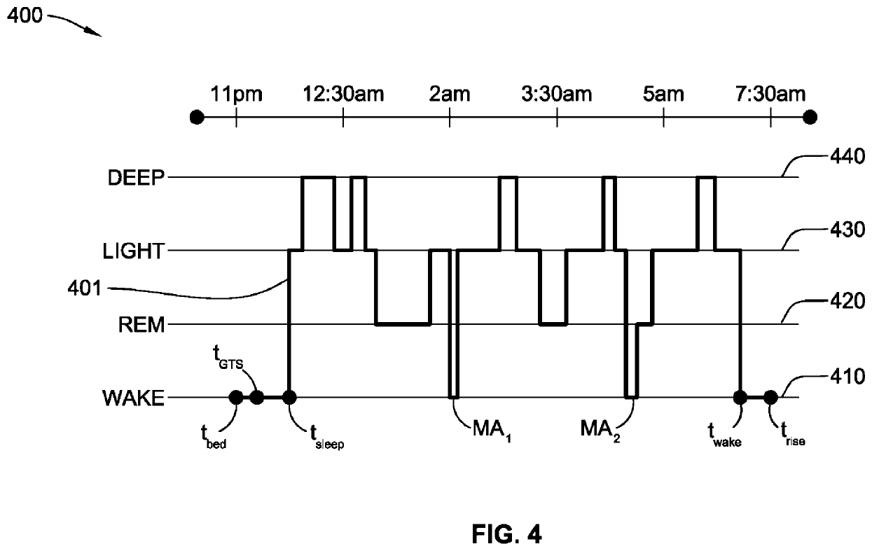

Referring to FIG. 4, an exemplary hypnogram 400 corresponding to the timeline 301 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (tGTS) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 4 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (tGTS), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (tGTS), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory therapy device 122, data indicative of the user donning the user interface 124, etc.), or any combination thereof.

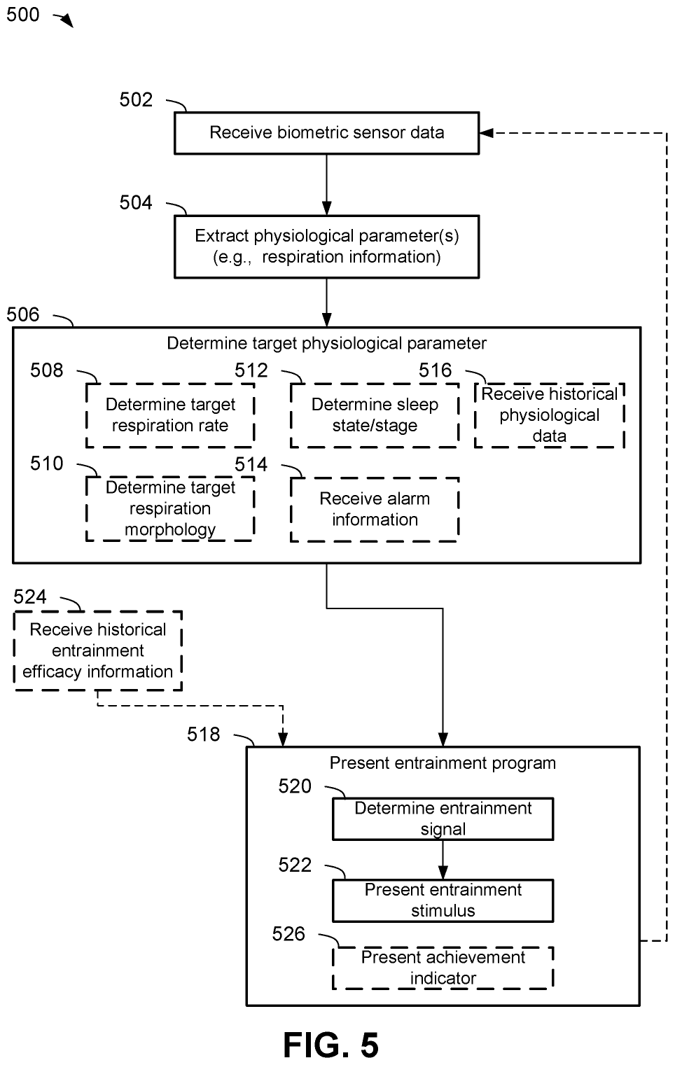

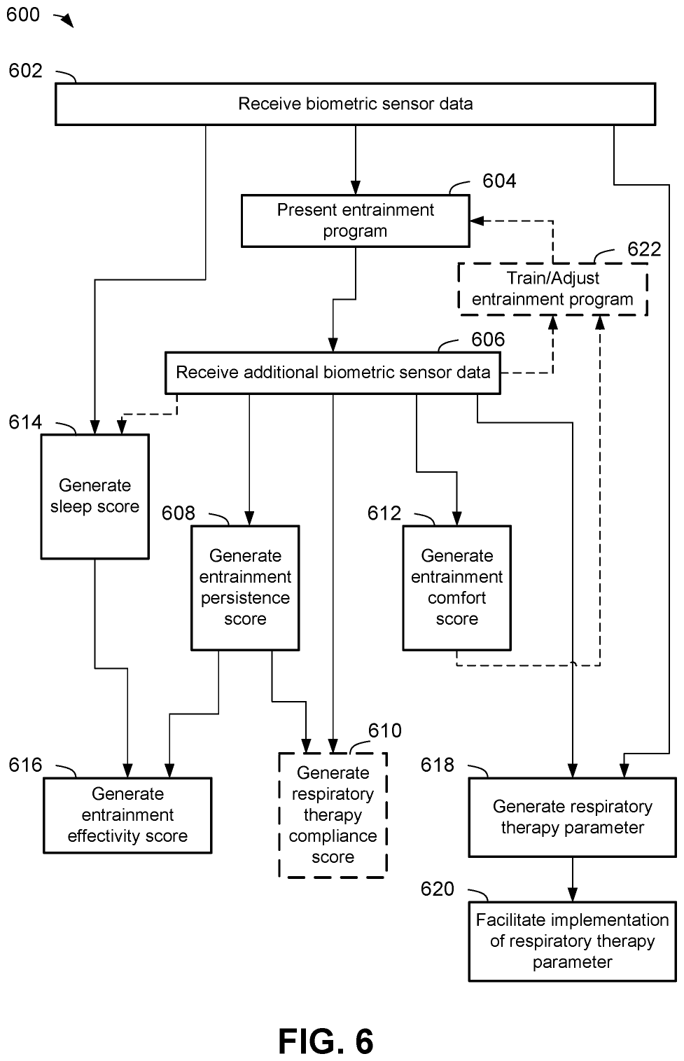

FIGS. 5-6 relate to presenting and using an intelligent entrainment program. An entrainment program as disclosed herein can be used by an individual to aid in falling asleep, remaining asleep, waking up, or otherwise. The intelligent entrainment program can be especially useful for individuals who have difficulty relaxing and falling asleep, especially those with increase sympathetic autonomic nervous system (ANS) activation in the time they wish to fall asleep (e.g., during a pre-sleep period). For individuals with difficulty falling asleep, if they are diagnosed with a sleep-related breathing disorder and prescribed respiratory therapy, the difficulties in falling asleep may lead to the individual being uncompliant with respect to the respiratory therapy, which can further harm the user's ability to achieve good sleep.

Because every individual has different natural breathing patterns and rhythms, as well as other individualized needs, certain “dumb” paced breathing tools have a low chance of success and/or a low chance of persistent success over time. In fact, use of a “dumb” paced breathing tool can increase stress for a user and produce counterproductive results. For example, a user of a “dumb” paced breathing tool may become frustrated when they are unable to achieve a desired pace or as the pacing signals become annoying, or the user may misperceive correlation of the user's breathing with the pacing signal. In such cases, the user may stop using the tool and may refuse to use the tool in the future. Additionally, if the tool is being used to prepare a user for respiratory therapy, such failures may lead the user to neglect respiratory therapy.

The intelligent entrainment program disclosed herein is capable of using physiological parameters of the individual to automatically adjust its entrainment program (e.g., the entrainment signal used and/or how the entrainment signal is presented, such as what entrainment stimuli are used) to the individual. In some cases, the intelligent entrainment program can instead or additionally learn the best entrainment parameters to use for a given individual. Additionally, certain aspects and features of the entrainment program can make use of and/or facilitate respiratory therapy.

In an example use case, a user may decide to go to sleep and may lay down in bed and begin an entrainment program via the user's smartphone. The user may place the smartphone on the nightstand next to the bed and start entrainment software. As the user lays down in bed, the software can receive biometric sensor data from one or more sensors of the smartphone and/or other sensors. Non-contact sensors, such as a SONAR sensor or the like, can be especially useful for the acquiring biometric sensor data for such an entrainment program because they would not interfere with the user's sleep or comfort the way a contacting sensor would. Additionally, such sensors can be easily incorporated into one or more devices that may regularly be at or near the user while the user is engaging in the entrainment program (e.g., before sleep). For example, a microphone-and-speaker-based SONAR sensor can be incorporated into a smartphone that is placed on a bedside table or incorporated into a bedside smart device to readily collect the desired biometric sensor data as the user engages in the entrainment program. Nevertheless, in some cases, contacting sensors can be used in addition to or instead of non-contact sensors. Physiological parameters can be extracted from the biometric sensor data and can be used to present the entrainment program (e.g., generate an entrainment signal and present an entrainment stimulus based on the entrainment signal). As a result, the user may be laying on the bed and may be breathing in an out in time with an audio stimulus emitted by a speaker on the smartphone. Using the user's physiological parameters, the audio stimulus (or other stimuli) provided by the entrainment software can be dynamically adjusted to best suit the individual. For example, to achieve an ultimate target respiratory rate, the entrainment software may monitor the user's current respiratory rate and present an entrainment signal that slowly changes from the user's current respiratory rate to the ultimate target respiratory rate. In another example, to achieve a desired respiratory morphology, the entrainment software may monitor the user's current respiratory morphology and present an entrainment signal that slowly changes from the user's current respiratory morphology to the desired respiratory morphology.

In another example use case, the same user may continue using the entrainment software while asleep. In such a case, the entrainment software may monitor the user's sleep state and/or sleep stage and adjust its entrainment program dynamically. For example, if the user is asleep and the entrainment software detects that the user is beginning to awaken prematurely, the entrainment software can provide an entrainment stimulus (e.g., an audio cue or adjusting the pressure/resistance of a respiratory therapy device used by the user) designed to keep the user asleep and/or move the user towards a target sleep state.

In another example use case, the same user may continue using the entrainment software up through waking. In such a case, the entrainment software may include an alarm function to wake the user at a particular time, within a particular window of time, after a predetermined length of sleep-related time (e.g., TIB, TST, PTST, total time in deep sleep, total time in REM sleep, etc.), after the conclusion of a particular sleep stage, or the like, or any combination thereof. Upon the occurrence of the triggering event (e.g., conclusion of a particular sleep stage after a preset alarm time), the entrainment software can present entrainment stimuli (e.g., an audio stimulus and a visual stimulus) designed to move the user's respiratory rate and/or morphology towards a respiratory rate and/or morphology associated with wakefulness.

While disclosed primarily in relation to sleep, aspects of the present disclosure can be used for other purposes, such as to control anxiety and/or hypertension. Certain aspects of the present disclosure can be used for meditation, such as to provide realtime feedback and goal-oriented evaluation of one or more meditation sessions.

The entrainment program can be used to entrain any physiological parameter to a target value. Often, the entrainment program is used to adjust a respiration pattern, such as a respiration rate and/or a respiration morphology (e.g., shape, rate, depth, and/or inspiration-expiration ratio of breath) of the user. In some cases, the entrainment program is used to adjust a breath path of the user (e.g., encourage nasal breathing). In some cases, the entrainment program is used to achieve a desired sympathetic or parasympathetic ANS parameter value.

For an example user breathing at approximately 0.1 Hz, a transition from a sympathetic to parasympathetic response is expected when falling asleep, where hyperarousal related to insomnia or other sleep disorders does not dominate. A tired but “wound up” state may lead to higher sympathetic activity than expected during NREM sleep, suggesting an unwanted awaking is more likely. In some cases, EEG activity (e.g., beta and gamma activity during NREM) can be compared with expected levels to identify whether the user may be moving towards an undesired wakening.

In some cases, the entrainment software can operate in an active mode or passive mode. In an active mode, the entrainment software can ask for and/or seek attention of the user to help focus the user on entrainment. In an active mode, the entrainment software can provide conspicuous stimuli to the user. An example of an active mode is the user actively selecting to perform a mediation, then concentrating on visual and audio cues provided by the entrainment software. In some cases, the user's focus or concentration can be tracked or monitored, such as by identifying how closely the user's respiratory rate tracks to the entrainment signal.

In a passive mode, the entrainment software can provide subtle or inconspicuous stimuli to the user. The passive mode can be known as an ambient mode. In the passive mode, the entrainment software can provide subtle stimuli that may not be explicitly noticed by the user as entrainment stimuli. For example, subtle stimuli can be provided by slightly altering, according to an entrainment signal, the pressure settings of a respiratory therapy device being used by the user. For another example, a subtle stimuli can be provided by slightly modulating, according to an entrainment signal, an audio stimulus (e.g., a song file) already being presented to the user.

Entrainment success can be monitored and evaluated in various fashions as disclosed in further detail herein, such as via sleep scores, entrainment persistence scores, entrainment comfort scores, and the like. In an example, one or more scores can be generated using physiological data indicative of how closely the user's breath morphology matched the desired breath morphology, how closely the user's breath path parameter matched the desired breath path parameter (e.g., nasal breathing), whether the user's breath sounds were indicative of congestion, whether the user's level of wakefulness and/or sleepiness changed in a desired fashion during the entrainment program, and the like.

Feedback from the level of entrainment success (e.g., one or more scores) can be used to train and/or tailor future entrainment programs. For example, a machine learning algorithm can be trained to maximize entrainment success based on certain set target variables (e.g., a target respiration pattern). In an example, such a machine learning algorithm (e.g., machine learning model) can be trained on breathing rates, breathing patterns, sleep onset times, stimuli effectiveness, other such parameters, or any combination thereof. In some cases, a machine learning algorithm can be trained to maximize one or more physiological parameters based on one or more other physiological parameters. For example, a machine learning algorithm can be trained to learn the depth and/or duration of inspiration, and optionally expiration, that achieves the most positive effect on parasympathetic innervation (e.g., via a parasympathetic ANS parameter).

In some cases, the level of entrainment success (e.g., one or more scores) can be provided to a sleep management system for further use, such as to a CBT-I system, a sleep improvement program, or a respiratory therapy management system. In some cases, the level of entrainment success can include comparing physiological parameters or one or more scores acquired during or after presentation of an entrainment program with similar physiological parameters or similar score(s) acquired before presentation of an entrainment program (e.g., a baseline).

Entrainment programs can be based on the received biometric sensor data (e.g., via the extracted physiological parameter(s)), including realtime or near realtime sensor data. By comparing to realtime and near realtime biometric sensor data and/or extracted physiological parameters (e.g., motion, heart rate, breathing rate, sympathetic nervous activation), the system can see if the current entrainment program (e.g., the current entrainment signal and/or the current route(s) of entrainment stimulus) is increasing or decreasing anxiety. The system can also process input data to adjust a target phase of an entrainment signal and/or add an offset to turning points in generation of an active or ambient stimulus (e.g., to change the shape of specific target breath morphologies). Such input data could also be used to adjust subtle features, and if undergoing respiratory therapy, to analyze the heart rate changes based on detection of cardiogenic oscillations (CGOs) and CGO beat times.

In some cases, the entrainment program can be used to practice entrainment prior to an intended use (e.g., a practice session prior to use during a pre-sleep period or during a sleep session). In some cases, a practice session can be used to practice achieving one or more physiological parameters while not necessarily needing to meet one or more other physiological parameters. In other words, the entrainment signal can be generated to entrain one or more target physiological parameters and to ignore or intentionally not entrain one or more other physiological parameters (e.g., one or more other physiological parameters that will be a target physiological parameter during an intended use session).

For example, a practice session can include practicing to achieve a particular style of breathing (e.g., breath morphology and/or breath path parameter) without necessarily worrying about the respiration pattern or without necessarily achieving the same respiration pattern that will ultimately be used in an intended use session. For example, in a practice session, the entrainment signal can be designed to entrain the user into a desired breath morphology (e.g., deep breathing) and/or breath path (e.g., nasal breathing), but will not attempt to entrain the user to a particular respiration pattern. In some cases, the entrainment signal can automatically adjust according to the user's current respiration pattern to avoid attempting to entrain the user to a given respiration pattern. For example, if the user's respiration rate starts to naturally increase or decrease during the training session, the entrainment signal can be dynamically modified to match or move closer to the user's new respiration rate.

In some cases, a user's first practice session may begin with an practice entrainment signal that is different from the ultimate entrainment signal used during the intended use, then may progressively move towards the ultimate entrainment signal over the course of the practice session or over the course of multiple practice sessions. For example, a first practice session may begin with the goal of reaching an entrainment signal of 10 breaths per minute, then progress over that same practice session or multiple practices sessions to a goal of an entrainment signal of 6 breaths per minute. In some cases, a similar entrainment signal progression may occur between multiple intended use sessions.

In some cases, a user's first practice session may begin with one or more practice entrainment stimuli that differ from the one or more ultimate entrainment stimuli used during the intended use, then may progressively move towards the one or more ultimate entrainment stimuli over the course of the practice session or over the course of multiple practice sessions. For example, a first practice session may begin with practice entrainment stimuli provided by visual and audio cues, whereas the ultimate entrainment stimuli may be provided by subtler audio cues and tactile cues. In some cases, a similar entrainment stimulus progression may occur between multiple intended use sessions.

In some cases, the entrainment program can especially focus on inspiration (e.g., inspiration rate, inspiration morphology, and the like). In some cases, a target rate at or around 0.1 Hz can be initially suggested. Training inspiration, as opposed to full breath or exhalation, and especially via nasal breathing, can be especially desirable to improve future compliance with respiratory therapy devices, and even more so for respiratory therapy systems that include nasal pillow masks. In contrast, having a breathing pattern that requires breathing in or out of the mouth is not desirable, as it may encourage mouth breathing, which can cause dryness—even when a full face mask is used during respiratory therapy—or discomfort (e.g., when a nasal pillow mask is used during respiratory therapy). Training inspiration via nasal breathing can also nudge the user to clear any congestion, such as using a saline spray, decongestant, anti-histamine and so forth.

Additionally, training on inspiration, and not setting expectations and/or parameters around expiration can allow the user to choose to relax during expiration rather than forced exhalation, which can be more natural and calming. During normal quiet (unforced) breathing, inspiration is an active process using muscles, whilst expiration is usually passive due to recoil, and is longer, followed by a pause. During inspiration, the increased volume leads to decreased intrapulmonary pressure (e.g., to around −1 cm H₂O). The pressure is lowest at mid inspiration, allowing air to be sucked in. During expiration, the pressure is increased (e.g., to around +1 cm H₂O (assuming atmospheric pressure is zero)). The pressure is highest at mid expiration. With obstruction (e.g., COPD), one may need to use expiratory muscles for forced expiration, or for restriction (e.g., for user suffering from fibrosis). Therefore, the system can recommend or suggest that the user decrease their intrapulmonary pressure, such that this pressure is lowest at a point defined in the program, and that the expiration is a passive process. Additionally, further reasoning for encouraging nasal breathing relates to the olfactory system and can include facilitating memory consolidation during entrainment.

FIG. 5 is a flowchart depicting a process 500 for presenting an entrainment program according to some implementations of the present disclosure. Process 500 can be performed by system 100 of FIG. 1, such as by a user device (e.g., user device 170 of FIG. 1). Process 500 can be performed in realtime or near realtime.

At block 502, biometric sensor data is received. The biometric sensor data can be received from one or more sensors, such as one or more sensors 130 of FIG. 1. The received biometric sensor data can include any suitable sensor data as disclosed herein, including, for example, heart rate data, temperature data, biomotion data (e.g., gross bodily movement data and/or respiration data), and the like. In some cases, biometric sensor data from one or more sensors can be used to synchronize additional biometric sensor data from one or more additional sensors. In some cases, physiological parameters identified from one or more channels of biometric sensor data at block 504 can be used to help synchronize the channels of biometric sensor data. In some cases, additional sensor data can be received at block 502, such as non-biometric sensor data.

In some cases, the biometric sensor data specifically includes biomotion data, such as biomotion data acquired via one or more non-contact sensors as disclosed herein. Biomotion data can relate to movement of the user during respiration and/or during a sleep session.

At block 504, one or more physiological parameters can be extracted from the received biometric sensor data. Extracting physiological parameters can include processing the received biometric sensor data. Extracting physiological parameters can include extracting respiratory information. In some cases, respiratory information can include i) respiratory rate, ii) respiration rate variability, iii) respiratory morphology, iv) inspiration amplitude, v) expiration amplitude, vi) inspiration-expiration ratio, vii) time of maximal inspiration, viii) time of maximal expiration, ix) length of time between breaths, x) forced breath parameter, xi) breath path parameter, xii) a change in any of the aforementioned parameters, or xiii) any combination of i-xii. Other similar respiratory-related parameters (e.g., respiratory information) can be extracted. A respiration pattern can refer to one or more respiratory-related parameters, such as any combination of one or more of i-xii identified above. The forced breath parameter can be a binary or non-binary parameter distinguishing between the user releasing breath and forcing exhalation. The breath path parameter can be a binary or non-binary parameter distinguishing between the user engaging in nasal breathing or mouth breathing. In some cases, respiratory information can be used to extract further physiological parameters.

In some cases, extracting respiration information can be based on biomotion sensor data. Biomotion information can be extracted from biometric sensor data. Chest movement information can be extracted from the biomotion information by processing the biomotion information. Respiration information can be determined by processing the chest movement information.

In some cases, extracting physiological parameters can include extracting other physiological parameters, such as a sympathetic ANS parameter and/or a parasympathetic ANS parameter. The sympathetic ANS parameter and parasympathetic ANS parameter can be parameters based on other physiological parameters, such as heart rate variability and galvanic skin response (GSR), that are indicative of sympathetic ANS activation and parasympathetic ANS activation, respectively. For example, an increase in heart rate variability and decrease in GSR can relate to an increase in parasympathetic innervation, and thus an increase in the parasympathetic ANS parameter. Since an increase in parasympathetic ANS activity can relate to a decrease of stress and movement towards a calm state suitable for sleep, it can be desirable to present an entrainment program that is most effective at increasing the parasympathetic ANS parameter during a pre-sleep period. As disclosed in further detail herein, the entrainment signal can be intelligently determined to most effectively adjust the desired physiological parameters for a given individual, such as an entrainment signal that most effectively increases the parasympathetic ANS parameter for the user.

In some cases, other physiological and/or non-physiological parameters can be extracted at block 504, such as non-physiological sleep-related parameters. Examples of non-physiological sleep-related parameters include parameters associated with a respiratory therapy device. For example, parameters extracted from sensor data received from a respiratory therapy device can be useful in extracting respiratory information.

At block 506, one or more target physiological parameters is determined. The target physiological parameter can be a physiological parameter that is to be adjusted through the process of entrainment, such as a respiratory pattern, which can include a respiratory rate and/or a respiratory morphology. Determining the target physiological parameter can include using the received biometric sensor data and/or extracted physiological parameter(s).

The target physiological parameter can be determined to achieve a given result. In an example, the target physiological parameter can be determined to promote sleep, to promote calming of the user's ANS, to promote a style of breathing (e.g., nasal breathing), or the like. In some cases, the target physiological parameter can be the end goal itself (e.g., a target number of breaths per minute). In some cases, however, the target physiological parameter can be a parameter that is correlated with the end goal (e.g., a target number of breaths per minute can be correlated with the goal of a desired level of parasympathetic ANS activation).

In some cases, determining the desired target physiological parameter can include determining a target respiration rate at block 508. Determining a desired respiration rate can include determining a desired rate of inspiration, such as six breaths per minute. The target respiration rate can be used as a target respiration pattern. In some cases, the target respiration rate can be a target inspiration rate.

In some cases, the target physiological parameters can make use of extracted physiological parameter(s) of the user from block 504. In an example, the target respiration rate determined at block 508 can be determined to be a respiration rate between a current respiration rate and an ultimate target respiration rate. For example, for a user breathing at 20 breaths per minute and an ultimate target respiration rate of 6 breaths per minute, the target respiration rate can be set to 15 breaths per minute. As the user approaches or meets the target respiration rate, the target respiration rate can be updated towards that of the ultimate target respiration rate. Thus, entrainment of a user's physiological parameter with a target physiological parameter can occur gradually through one or more intermediate stages. The target physiological parameter(s) at such intermediate stages can be considered intermediate target physiological parameter(s).

In some cases, determining a target physiological parameter at block 506 can include determining a target respiration morphology at block 510. The target respiration morphology can be a desired respiration morphology that is based on the user's current physiological parameters. The target respiration morphology can be used as a target respiration pattern. In some cases, the target respiration morphology can be a target inspiration morphology.

In some cases, determining a target physiological parameter at block 506 can include determining a sleep state and/or sleep stage at block 512. The sleep state and/or sleep stage determined at block 512 can be determined based on the extracted physiological parameter(s) of block 504. The target physiological parameter can be different for the user depending on whether the user is awake, asleep, in a light sleep, in a deep sleep, in REM sleep, or otherwise. In some cases, the target physiological parameter can be dependent on time spent in one or more sleep stages or sleep states, and/or dependent on a pattern of subsequent sleep stages or sleep states.

In some cases, determining a target physiological parameter can include receiving alarm information at block 514. Receiving alarm information can include receiving information about when the alarm should trigger, such as receiving an alarm time, an alarm window (e.g., period of time in which the user is to be wakened), a predetermined length of sleep-related time (e.g., TIB, TST, PTST, total time in deep sleep, total time in REM sleep, etc.) desired before an alarm is to be issued, a desired sleep stage in which the alarm is to be issued, a desired sleep stage in which no alarm is to be issued, or the like, or any combination thereof. The alarm information can define a trigger. The trigger can be multi-part. For example, a trigger can require the current time to be past a preset alarm time and the user to be in a certain sleep stage. Once the trigger occurs (e.g., the triggering event occurs), the system can set the target physiological parameter to one associated with the alarm. For example, an alarm for waking a user can involve setting the target physiological parameters to one associated with wakefulness, such as a respiration rate at or above a threshold respiration rate.

In some cases, if the alarm's trigger conditions have not been met and the system detects that the user is beginning to awaken, the system can automatically determine a target physiological parameter designed to keep the user from waking prematurely. For example, when the system detects that the user is moving from N1 to N2, the system can select a target physiological parameter that is designed to encourage the user to move back to N2 from N1.

In some cases, determining the desired target physiological parameter can include accessing historical physiological data at block 516. Such historical physiological data can include historical sleep-related physiological data. Historical physiological data can be used to recreate a past experience that has been effective or desirable. For example, if a certain breathing pattern (e.g., respiratory rate(s) and/or respiratory morphology(ies)) was effective at helping a user fall asleep in the past, the target physiological parameters can be established to achieve that breathing pattern. In such an example, historical physiological data from one or more previous sleep sessions can be analyzed to determine a pattern of respiratory rates that resulted in consistently low sleep onset times, then the target physiological parameter can be determined to move the user through that pattern of respiratory rates. Other techniques for evaluating the effectiveness of entrainment and/or for otherwise evaluating the user's sleep, such as disclosed in further detail herein, can be used to identify appropriate physiological parameters to use for a target physiological parameter. In some cases, historical entrainment efficacy information can include information related to respiration patterns achieved after certain entrainment stimuli are presented, indirect effects of presented stimuli, sleep onset latency, wake after sleep onset, sleep fragmentation, and the like.

In some cases, the target physiological parameter can be based on a physiological parameter associated with the user falling asleep in the past. For example, one or more historical respiratory rates achieved by the user when falling asleep during one or more previous sleep sessions can be used to define a target respiratory rate.

In some cases, determining the target physiological parameter at block 506 can involve using other physiological or non-physiological parameters. For example, medical record information and/or respiratory therapy information (e.g., from a respiratory therapy device) can be used to identify that the user makes use of a respiratory therapy device. In such cases, it may be especially beneficial to encourage nasal breathing instead of mouth breathing, especially if the respiratory therapy device is paired with a nasal pillow mask. When such a determination is made, determining the target physiological parameter at block 506 can include setting a target breath path parameter to a value that would encourage nasal breathing.

At block 518, an entrainment program can be presented. Presenting an entrainment program at block 518 can include determining an entrainment signal at block 520 and presenting an entrainment stimulus based on the entrainment signal at block 522.

Determining an entrainment signal at block 520 can include determining a signal (e.g., a waveform, such as a breathing waveform) that can be used to entrain the user's respiratory actions towards desired respiratory actions to ultimately achieve the determined target physiological parameter from block 506. Determining the entrainment signal at block 520 uses the determined target physiological parameter from block 506.

In some cases, the entrainment signal determined at block 520 can be representative of an inhalation and/or exhalation pattern or rhythm. For example, for a given target respiration rate determined at block 508, the entrainment signal can repeat at the same frequency as the respiration rate (e.g., to encourage respiration at the respiration rate). As the target respiration rate changes, the entrainment signal can change. As another example, for a given target respiration morphology from block 510, the entrainment signal can fluctuate in a correlated fashion that matches the fluctuations of the respiration morphology (e.g., respiration morphology indicating a fast-then-slowing inspiration shape can result in an entrainment signal that quickly-then-slowly increases).

In some cases, the entrainment signal can be determined at block 520 based on one or more physiological parameter(s) determined at block 504. For example, the entrainment signal can be based on a lung capacity parameter extracted from the biometric sensor data from block 502. A lung capacity parameter can be obtained from a spirometer or other sensor. In some cases, a lung capacity measurement (e.g., lung capacity parameter) can be determined from a sensor other than a spirometer, such as a microphone, that has been previously calibrated using one or more previous measurements of the user's lung capacity acquired from a spirometer. An entrainment signal customized to lung capacity can have its amplitude adjusted according to the user's individual lung capacity. Thus, while “dumb” paced breathing tools might provide a signal that continues inspiration after the user's lung capacity has been met, certain aspects of the present disclosure can automatically adjust the entrainment signal based on the user's lung capacity. In some cases, adjustment of the entrainment signal can occur before the entrainment stimulus is presented to the user at block 522, although that need not be the case. In some cases, for example, the system can actively monitor how close the user is to a full inspiration or full expiration and actively adjust the entrainment signal in realtime or near realtime to more closely match the user's lung capacity. The degree to which the entrainment signal is adjusted based on a physiological parameter (e.g., lung capacity) can be unique to each individual and can be learned over time to maximize entrainment efficacy, sleep quality, or the like.

At block 522, one or more entrainment stimuli can be presented based on the entrainment signal from block 520. Presenting an entrainment stimulus can include generating a stimulus, according to the entrainment signal, using any suitable stimulus device. For example, presenting an entrainment stimulus can include presenting and/or modulating audio sounds, such as modulating the sound of ocean waves according to the pattern of the entrainment signal. In another example, presenting an entrainment stimulus can include adjusting the pressure relief settings of a respiratory therapy device according to the entrainment signal.

Presenting an entrainment stimulus can include presenting the entrainment signal, a portion of the entrainment signal, or information based on the entrainment signal. Presenting an entrainment stimulus can include presenting any suitable stimulus that is conspicuously (e.g., in an active mode) or inconspicuously or subtly (e.g., in an active mode or a passive mode) discernable to the user. Any suitable stimulus device can be used.

In some cases, presenting an entrainment program at block 518 can include determining whether to present entrainment stimulus in an active mode or a passive mode. In some cases, determining whether to present the entrainment stimulus in an active mode or a passive mode can occur in response to intentional user input, such as actuation of a button on a GUI.

In some cases, whether or not to present the entrainment stimulus in an active mode or a passive mode can occur automatically in response to received biometric sensor data (e.g., in response to extracted physiological parameter(s), such as galvanic skin response, heart rate variability, blood pressure, biomotion, or any combination thereof). When changing modes automatically, the system can automatically identify when the user would benefit more from an active mode or a passive mode, then automatically switch modes. For example, while some users may achieve better results when being told to actively focus on entrainment, that same approach may be detrimental to other users. Likewise, situations may arise where a single user may benefit more from an active or passive mode of entrainment than from the other mode of entrainment.

In an example, for a user receiving entrainment stimuli in an active mode, the system may use extracted physiological parameters to identify that the user is starting to become annoyed or that the user is starting to fall asleep, then the system may make a determination that the user would benefit from receiving entrainment stimuli in a passive mode instead (e.g., to avoid annoying the user and harming future compliance, or to avoid waking or rousing the user) and switch modes accordingly. In another example, for a user receiving entrainment stimuli in a passive mode, the system may use extracted physiological parameters to identify that the user is in a calm state or ready to awaken, in which case the system may make a determination that the user would benefit from receiving entrainment stimuli in an active mode instead (e.g., to provide more engaging entrainment or to aid in waking or rousing the user) and switch modes accordingly.

In some cases, the system can identify that the user may benefit from entrainment stimuli provided in an active mode and can provide a notification to the user to request permission before switching modes. For example, the system can use extracted physiological parameters to identify that the user is having difficulty falling asleep (e.g., at the beginning of a sleep session or at a point of wakefulness mid-sleep-session), then the system can notify the user by presenting a message such as “It looks like you are having trouble falling asleep. Can we help? Press “OK” to begin an entrainment session.” If the user accepts, the system can start and/or switch to an active mode to provide entrainment stimuli.

In some cases, the system can identify that the user may benefit from an entrainment program when the user is not currently engaging an entrainment program. For example, the system can use extracted physiological parameters (e.g., extracted sleep-related parameters, such as a sleep onset latency) to identify that the user is having difficulty falling asleep or has been having difficulty falling asleep for a series of sleep sessions (e.g., the past few nights). Based on this identification, the system can begin presenting an entrainment program, whether automatically (e.g., without further user action) or after acknowledgement by the user in response to a suggestion to start an entrainment program (e.g., a user indicating “Yes” in response to the system presenting a prompt to start an entrainment program).

Examples of entrainment stimuli include presenting an audio stimulus, modulating an existing audio stimulus, presenting a visual stimulus, modulating an existing visual stimulus, presenting a tactile stimulus, modulating an existing tactile stimulus, or the like. In some cases, multiple types of entrainment stimuli can be used in combination, although that need not always be the case. In some cases, multiple entrainment signals can be determined (e.g., generated) at block 520, and different entrainment stimuli can be presented based on the different entrainment signals.

In some cases, entrainment stimuli can include one or more audio stimuli (e.g., specialized sounds, user elected sounds, random sounds, or the like) which may be background or masking, or modulated based on the entrainment signal (e.g., at a rate consistent with a desired personalized breathing pattern). Examples of audio stimuli include audio output from any suitable transducer, such as speakers (e.g., headphones, smartphone speakers, pillow speakers, speakers incorporated into a respiratory therapy system's user interface, and the like), bone conduction devices (e.g., bone conduction headphones), or other audible devices (e.g., sound from a flow generator of a respiratory therapy device, sounds from a vibration motor, or the like).

In some cases, entrainment stimuli can include one or more visual stimuli, such as direct illumination, ambient illumination (e.g., modulated glow), projection of a scene or visualization, projection a hologram, or any combination thereof. Examples of visual stimuli include graphics displayed on a display device (e.g., a trace on a screen that follows the entrainment signal), lights emitted using a light emitting device (e.g., a light emitting diode), lighting controlled via a remote device (e.g., controlling a networked light bulb or light switch or controlling a networked display device), and the like. Visual stimuli can include modulating an existing visual stimulus, such as changing the intensity or color temperature of one or more lights, or changing a projection on a screen or a presented hologram.

In some cases, entrainment stimuli can be tactile stimuli, such as a vibration stimulus or actuation of some other physical actuator, either worn or separate (e.g., a smartphone modulated actuator). Tactile stimuli can be especially useful when a bed partner might otherwise be disturbed by sounds or light in the room. Examples of tactile stimuli include vibrations, taps, and other physically discernable stimuli, which can be provided by wearable devices, surfaces (e.g., a mattress or a pillow with a stimulus device), and the like. In some cases, a tactile stimulus can include modulating a physical property of a physical material (e.g., inflating and deflating a pillow or mattress to change its firmness). In some cases, a tactile stimulus can be respiratory-related, such as controlling the amount of expiratory pressure relief provided by a respiratory therapy device. For example, an entrainment stimulus can include adjustment of one or more parameters of a therapy device being used by the user, such as a respiratory therapy device. In such an example, the entrainment stimulus can be modulation of the operation of the flow generator.

Other types of stimuli can be provided, such as via taste or scent (e.g., controlling release of a substance, such as from receptacle 180 of FIG. 1).

Different types of stimuli can be more effective for different individuals and/or for the same individual at different times. As an example, some users may prefer or respond better to listening to modulated soundscapes (e.g., modulated ocean wave sounds), whereas other prefer to listen to counting (e.g., “1, 2, 3 . . . and out”). Likewise, some users may prefer or respond better to visual stimuli (e.g., illumination or modulation of room lights, device lights, smartphone screen, respiratory therapy device lights/screen, or the like). In some cases, some uses may prefer or respond better to listening to live sounds (e.g., sounds from a fan in the room or from the flow generate of a respiratory therapy device) rather than pre-recorded sounds. Thus, presenting an entrainment stimulus at block 522 can include selecting one or more appropriate types of stimuli to present, such as based on user pre-defined preferences, user feedback, or analysis of one or more physiological parameters of the user.

In some cases, the entrainment stimulus is selected to not interfere with the sensors used for acquiring the received biometric sensor data from block 502. In some cases, the entrainment stimulus may be selected to be outside of a range of sensing of the one or more sensors used to acquire the received biometric sensor data from block 502. For example, a visual sensor may operate in an infrared spectrum, while a visual stimulus presented at block 522 may be selected to present light outside of the infrared spectrum.

In some cases, the received biometric sensor data from block 502 is received from one or more sensors on a first device and the entrainment stimulus is selected to be provided by a second device that is different from (e.g., separate from) the first device. For example, in some cases the received biometric sensor data includes biomotion data acquired using audio sensors (e.g., microphone(s) and speaker(s)) on a smartphone. In such cases, the entrainment stimulus can be presented by a second device that is separate from the smartphone, such as a respiratory therapy device, a wearable device, a wired or wireless remote speaker (e.g., controlled by the smartphone or by another audio source, such as a wired or wireless pillow speaker), or the like. Thus, the user may receive audio stimulus (or other stimulus) without compromising the ability for the audio sensor(s) of the smartphone to collect the biomotion data.

In some cases, presenting an entrainment program at block 518 can include adjusting settings of a respiratory therapy device. For example, when the determined target physiological parameter and/or determined entrainment signal would require the user to perform longer-than-usual inhales, the system can adjust a setting (e.g., adjustable parameter) of a respiratory therapy device to permit longer inhales. In some cases, presenting the entrainment stimulus at block 522 can include adjusting one or more settings of a respiratory therapy device to effect the stimulus. For example, the expiratory pressure relief (EPR) setting can be adjusted.

In some cases, presenting an entrainment program at block 518, and optionally presenting an entrainment stimulus at block 522, can include presenting a supplemental stimulus that may or may not be based on the entrainment signal. The supplemental stimulus can be used to entrain and/or otherwise affect one or more additional physiological parameters. For example, in some cases a supplemental stimulus can be provided to encourage the user engage in nasal breathing, as discussed in further detail herein.

In some cases, presenting the entrainment program at block 518 can include presenting an achievement indicator at block 526. An achievement indicator can be a stimulus indicative of how close the user is to achieving the target physiological parameter. Any suitable achievement indicator can be provided, such as a visual indicator, a tactile indicator, an audio indicator, or the like. Despite what entrainment stimulus is provided at block 522, an achievement indicator can be a separate stimulus that shows the user's progression towards the target physiological rate. For example, a user with a target respiratory rate of 10 BPM that starts entrainment while at a respiratory rate of 20 BPM can currently have a respiratory rate of 15 BPM that is slowly moving towards 10 BPM as the user is engaging in the entrainment program. In such an example, a display device may present a visual achievement indicator, such as a circle of light that changes from red at 20 BPM to orange at 15 BPM and to green at 10 BPM. In some cases, the achievement indicator can be integrated with the entrainment stimulus provided at block 522. For example, a speaker playing an audio signal may modulate in a first fashion (e.g., by moving repeatedly between a first frequency limit and a second frequency limit) to indicate the entrainment stimulus and may modulate in a second fashion (e.g., increasing or decreasing the first frequency limit and/or second frequency limit) to indicate the achievement indicator. Similarly, a visual entrainment stimulus may change color (e.g., red to (orange to) green) as described above as the user's respiration pattern approaches the target respiration pattern or may change color (e.g., orange to red) as the user's respiration pattern diverges from the target respiration pattern.

In some cases, the physiological parameter indicated by the achievement indicator is the same type of physiological parameter as the target physiological parameter from block 506. For example the achievement indicator presented at block 526 can be based on respiration pattern as the entrainment program is entraining the user to achieve a target respiration pattern. In some cases, however, the physiological parameter indicated by the achievement indicator is a separate physiological parameter different form the target physiological parameter from block 506. For example, while an entrainment program may entrain a user to achieve a target respiratory rate, the achievement indicator may indicate a progression of a separate physiological parameter, such as a parasympathetic ANS parameter, towards a target for that parameter.

In some cases, historical entrainment efficacy information can be received at block 524 and used in presenting the entrainment program at block 518. The historical entrainment efficacy information can be indicative of efficacy of presentation of one or more past entrainment programs (e.g., efficacy of presentation of one or more past entrainment stimuli). The historical entrainment efficacy information can be received from local or remote data sources. In some cases, historical entrainment efficacy information can include historical entrainment program information (e.g., data, settings, and/or parameters used to present a past entrainment program), historical biometric sensor data, historical physiological parameters, historical sleep scores, historical entrainment persistence scores, historical entrainment comfort scores, historical entrainment effectivity scores, and the like. Based on knowledge of past entrainment programs that have been presented to the user (individualized historical entrainment efficacy information) or to other users having similar demographic information (demographic historical entrainment efficacy information), an entrainment program can be presented (e.g., an entrainment signal can be generated and an entrainment stimulus can be presented).

In some cases, process 500 can repeat by continuing to receive biometric sensor data at block 502. While the blocks of process 500 are depicted in a certain order, some blocks can be removed, new blocks can be added, and/or blocks can be moved around and performed in other orders, as appropriate.

FIG. 6 is a flowchart depicting a process 600 for using an entrainment program according to some implementations of the present disclosure. Process 600 can be performed alongside and/or as part of process 500. For example, blocks 602 and 604 can be similar to and/or the same as blocks 502 and 518 of FIG. 5.

At block 602, biometric sensor data is received, similar to block 502 of FIG. 5. At block 604, an entrainment program is presented, similar to block 518 of FIG. 5. Presenting the entrainment program at block 604 can include initially presenting an entrainment program (e.g., starting presentation of an entrainment program for a first time); presenting a full entrainment program (e.g., from a start time to and end time); or presenting a portion of an entrainment program (e.g., continuing or resuming an ongoing entrainment program, such as where multiple iterations of presenting an entrainment program are combined to completely present the full entrainment program from start to end). In some cases, an entrainment program presented in a pre-sleep period can be presented, and optionally modified, while the user is sleeping or otherwise engaging in the sleep session.

At block 606, additional biometric sensor data can be received. Additional biometric sensor data can be received similarly to block 602. In some cases, receiving additional biometric sensor data is merely an additional iteration of block 602.

In some cases, receiving the biometric sensor data includes receiving biometric sensor data associated with a user prior to presentation of an entrainment program, whereas receiving the additional biometric sensor data includes additional receiving biometric sensor data associated with a user during and/or after presentation of an entrainment program (or multiple entrainment programs, such as at least a threshold number of entrainment programs). Thus, comparison of the biometric sensor data and the additional biometric sensor data can provide information usable to compare factors before and during and/or before and after presentation of an entrainment program.

After an entrainment program is initially presented (e.g., after presentation of an entrainment program begins), an entrainment persistence score can be generated at block 608. The entrainment persistence score can be an indication of how closely a user followed, or is currently following (e.g., if being monitored in realtime), the entrainment program, such as how closely the user's physiological parameters are entrained to the target physiological parameters, how long the user engages with the entrainment program (e.g., as determined by the user's physiological parameters and analysis thereof to identify if the user appears to be engaging the entrainment program), how often the user attempts to use the entrainment program, and the like. Generating an entrainment persistence score can be based at least in part on the biometric sensor data and/or the additional biometric sensor data. In some cases, the entrainment persistence score can be based at least in part on i) a difference between a respiration pattern of the user and the target respiration pattern, ii) a rate of change of the respiration pattern, iii) a length of time the respiration pattern remains within a threshold of the target respiration pattern, iv) a length of time the rate of change of the respiration pattern remains within a rate of change threshold, or v) any combination of i-iv. In some cases, the entrainment persistence score is based on one or more similarity or dissimilarity indexes. A similarity index and a dissimilarity index can be calculated to determine the closeness (e.g., close or not close, respectively) of two parameters (e.g., a current parameter and a target parameter).

In some cases, after an entrainment program is initially presented, a respiratory therapy compliance score can be generated at block 610. The respiratory therapy compliance score can be indicative of how compliant the user is expected to be at respiratory therapy based on biometric sensor data and/or additional biometric sensor data collected with reference to presentation of an entrainment program. For example, the respiratory therapy compliance score can be indicative of a likelihood to comply with a respiratory therapy program that includes use of a removable user interface (e.g., a nasal pillow mask) to deliver respiratory therapy. The respiratory therapy compliance score is based at least in part on at least one determined physiological parameter, such as those described herein. In some cases, the respiratory therapy compliance score is based at least in part on the entrainment persistence score and determined sleep quality information (e.g., sleep-related parameters).

In some cases, after an entrainment program is initially presented, an entrainment comfort score can be generated at block 612. The entrainment comfort score can be based on subjective feedback from the user and/or physiological data collected from the biometric sensor data and/or additional biometric sensor data. For example, in some cases a user can provide subjective feedback indicative of a level of comfort (e.g., tapping on a rating from 1 to 5 of how comfortable the user feels), and the entrainment comfort score can be based on that subjective feedback. In some cases, the comfort score can be based on biometric sensor data and/or additional biometric sensor data, such as based on physiological parameter(s) indicative of a user's subjective level of comfort. For example, a parasympathetic ANS parameter can be indicative of a degree of comfort. Therefore, a comfort score comparison between pre- and post-entrainment program presentation can be made by comparing the change in parasympathetic ANS parameter from before presentation of the entrainment program and during and/or after presentation of the entrainment program. In some cases, the entrainment comfort score is based on a comparison between a comfort score from before presentation of the entrainment program and a comfort score from during and/or after presentation of the entrainment program.

In some cases, after an entrainment program is initially presented, a one or more respiratory therapy parameters can be generated at block 618. Generation of the one or more respiratory therapy parameters can include using one or more extracted physiological parameters from biometric sensor data and/or additional biometric sensor data. The respiratory therapy parameters can be one or more settings of a respiratory therapy device (e.g., flow generator settings) and/or other respiratory therapy parameters (e.g., choice of user interface type). The respiratory therapy parameters can be determined to provide the most expected efficacy and/or highest expected compliance based on the biometric sensor data and/or additional biometric sensor data associated with presentation of an entrainment program. In some cases, the respiratory therapy parameter(s) can be generated before the user ever uses a respiratory therapy device, in which case the parameter(s) can be used to set up the respiratory therapy device for a first time. In other cases, the respiratory therapy parameter(s) can be used to adjust current respiratory therapy parameter(s) of a respiratory therapy device that the user already uses.

In some cases, generation of the respiratory therapy parameter at block 618 can make use of one or more scores, such as the entrainment persistence score of block 608 and/or the respiratory therapy compliance score of block 610.

In some cases, after a respiratory therapy parameter is generated at block 618, implementation of the respiratory therapy parameter can be facilitated at block 620. Facilitation of a respiratory therapy parameter can be manual or automatic. Manual facilitation can include presenting (e.g., presenting on a display device) the respiratory therapy parameter to the user and/or a healthcare professional for reference when adjusting settings of a respiratory therapy device. Automatic facilitation can include transmitting the respiratory therapy parameter(s) to a respiratory therapy device to automatically adjust setting(s) of the respiratory therapy device.

In some cases, at block 614, a sleep score can be generated. The sleep score can be any suitable score indicative of a quality of sleep or any other evaluation of sleep. Generation of the sleep score can be based on one or more physiological parameters extracted from the biometric sensor data and/or the additional biometric sensor data. In some cases, a sleep score from before presentation of the entrainment program can be compared with a sleep score from during and/or after presentation of the entrainment program to determine a change in sleep score between before, during, and/or after initial presentation of the entrainment program. In some cases, a sleep score can be generated from sleep-related physiological parameters as an indication of quality of sleep. The sleep score can be based on various sleep-related physiological parameters, such as total time in bed, total sleep time, ratio of total sleep time to total time in bed, compliance with target go-to-bed time and/or get-out-of-bed time, number of detected events, number and/or type of sleep stages, time spent in certain sleep stages, movement detected during the sleep session, and the like.

In some cases, an entrainment effectivity score can be generated at block 616. The entrainment effectivity score can be indicative of an effectiveness of the entrainment program at improving sleep quality or sleep-related factors. In some cases, the entrainment effectivity score is based on a sleep onset time (e.g., the lower the sleep onset time, the more effective the entrainment program is assumed to be).

In some cases, generation of the entrainment effectivity score can be based at least in part on the sleep score generated at block 614 and/or the entrainment persistence score from block 608. For example, if a high sleep score (e.g., a positive change from before-to-after entrainment program presentation) occurs along with a high entrainment persistence score, the two scores can indicate that the user is achieving improved sleep after having engaged with the entrainment program. On the other hand, a low entrainment persistence score may indicate that the entrainment program did not have a strong impact on whatever the sleep score may be.

In some cases, the entrainment program can be trained and/or adjusted at block 622. Training and/or adjusting the entrainment program at block 622 can include training one or more machine learning algorithms and/or adjusting one or more settings or parameters associated with the entrainment program. Training and/or adjusting the entrainment program can include using the additional biometric sensor data, and optionally the biometric sensor data. In some cases, training and/or adjusting the entrainment program can make use of one or more scores, such as a sleep score from block 614, an entrainment effectivity score from block 616, an entrainment persistence score from block 608, a respiratory therapy compliance score from block 610, an entrainment comfort score from block 612, or any other score. Training and/or adjusting the entrainment program can include training an algorithm and/or adjusting one or more settings over multiple iterations to maximize one or more physiological parameters and/or one or more scores.

In some cases, training the entrainment program can include learning one or more physiological parameters (e.g., the depth and duration of inspiration) that has the most positive effect on an end goal (e.g., a physiological parameter, such as a parasympathetic ANS parameter). Thus, in future instances of presenting the entrainment program 604, the entrainment program will be presented in a fashion that entrains to the learned physiological parameter(s). Such learning can be updated through multiple iterations of block 622.

Examples of other physiological parameters that may be especially useful include breath path parameter (e.g., a nasal airflow rate or other indication of nasal breathing), a combination of breath path parameter and depth of inspiration, a hold time prior to exhalation, and the like. The depth and duration of the inhale can be tailored to a desired comfort level. For example, for some individuals, the entrainment program can present a more difficult goal (e.g., presenting the ultimate target depth and duration of inhale, rather than working slowly to the ultimate target depth and duration of inhale) to encourage the user to bring more focus to breathing, potentially excluding other factors (e.g., worry, stress, or other distractions). For some individuals, the entrainment program that is learned to be most desired (however measured) may present a less difficult goal (e.g., presenting intermediate targets between a current physiological parameter and an ultimate target physiological parameter). In an example, an entrainment program could include an initial focus period followed by a gradual reduction in the difficulty in sustaining this program (e.g., a mix of expected and detected habituation of the person, as well as adjustments in the program). A harder aspect could be a deeper and/or longer duration inhale—and to focus on nasal breathing.

In some cases, a default trained algorithm (e.g., machine learning algorithm) and/or default settings can be established based on the user's demographic information and/or other known or assumed information about the user prior to presentation of the entrainment program. Then, as the user makes use of the entrainment program (e.g., repeated iterations of blocks 604 and 622), the algorithm can be trained and/or the settings can be adjusted to be tailored to the individual.

In some cases, additional iterations of block 604 can include using one or more scores (e.g., a sleep score from block 614, an entrainment effectivity score from block 616, an entrainment persistence score from block 608, a respiratory therapy compliance score from block 610, an entrainment comfort score from block 612, or any other score) to determine the entrainment signal and/or present the entrainment stimulus. For example, if a generated comfort score decreases below a threshold, a future iteration of presentation of an entrainment program at block 604 can include using a more gentle entrainment signal or entrainment stimulus to try and increase the comfort score.

While the blocks of process 600 are depicted in a certain order, some blocks can be removed, new blocks can be added, and/or blocks can be moved around and performed in other orders, as appropriate. For example, in some cases, process 600 might include repeated iterations of blocks 602, 604, and 622 (e.g., with a future iteration of receiving biometric sensor data at block 602 occurring instead of receiving additional biometric sensor data at block 606). In another example, process 600 might include generating the entrainment persistence score at block 608, followed by generating the respiratory therapy compliance score at block 610, and then followed by generating the respiratory therapy parameter at block 618 based on the entrainment persistence score.

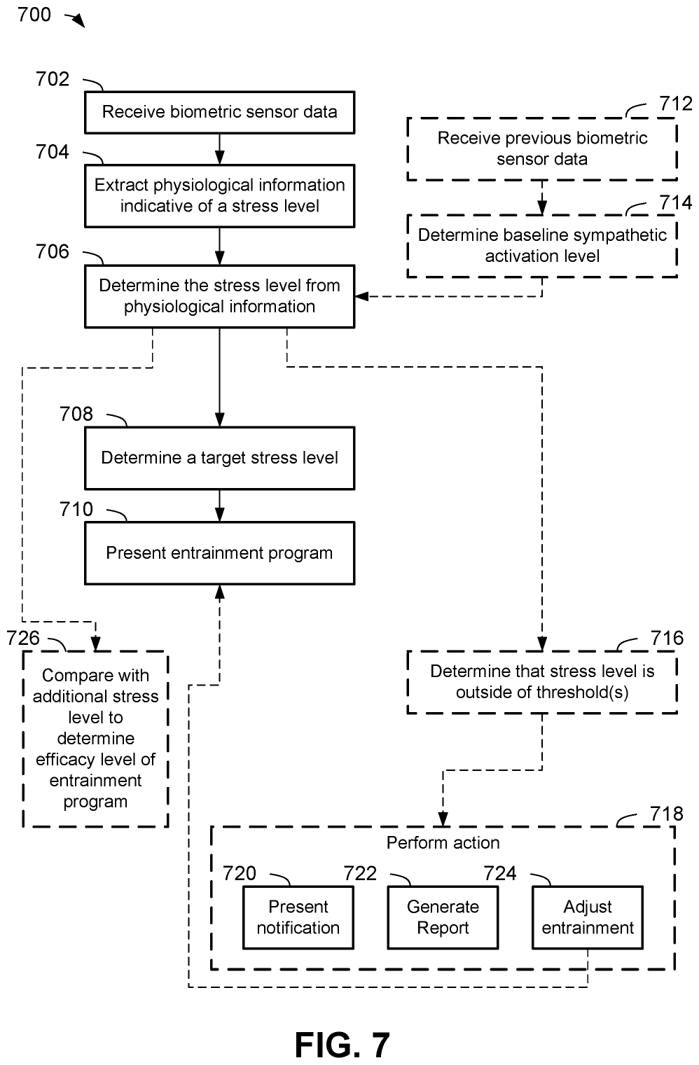

FIG. 7 is a flowchart depicting a process 700 for presenting an entrainment program based on stress level according to some implementations of the present disclosure. Process 700 can be similar to process 500 of FIG. 5—especially with respect to blocks 702, 704, 708, 710 of FIG. 7 and blocks 502, 504, 506, 518 of FIG. 5, respectively—except based specifically on a stress level instead of target physiological parameters. In some cases, a stress level as used with respect to process 700 can nevertheless be considered a physiological parameter as used with respect to process 500, although that need not always be the case.

At block 702, biometric sensor data is received. Receiving biometric sensor data at block 702 can be similar to receiving biometric sensor data at block 502 of FIG. 5. Any suitable biometric sensor data can be received, from any number of sensors (e.g., one or more sensor(s) 130 of FIG. 1).

In some cases, the received biometric sensor data includes motion sensor data acquired by a motion sensor. This motion sensor data can be indicative of motion of a user, such as while a user is walking around during the day or while the user is sleeping, or while the user is attempted to get to sleep. In some cases, the motion sensor can include i) an accelerometer, ii) a sonar sensor, iii) a radar sensor, or iv) any combination of i-iii. Any other suitable motion sensor can be used, such as a camera.

In some cases, the received biometric sensor data includes photoplethysmography (PPG) data acquired by a PPG sensor. Any suitable PPG sensor can be used, such as one incorporated in a HSAT. In some cases, the received biometric sensor data includes electrodermal activity level such as galvanic skin response (GSR) data acquired by a GSR sensor. Any suitable GSR sensor can be used, such as one that may be incorporated in a HSAT. In some cases, the received biometric sensor data includes blood pressure sensor data. The blood pressure sensor data can be acquired in any suitable fashion, such as via a blood pressure monitor (e.g., blood pressure device 182 of FIG. 1).

In some cases, the biometric sensor data includes i) electroencephalogram (EEG) data, ii) electromyogram (EMG), iii) electrooculogram (EOG), iv) electrocardiogram (ECG or EKG) data, or v) any combination of i-iv. EEG data and ECG data can be acquired from any suitable sensors (e.g., EEG sensor 158 and ECG sensor 156 of FIG. 1, respectively).

At block 704, physiological information indicative of a stress level can be extracted from the biometric sensor data. Physiological information indicative of a stress level can be any physiological data that can be used to determine or infer a stress level of the user. In some cases, the physiological information extracted at block 704 includes i) a respiration rate, ii) heart rate, iii) heart rate variability, iv) user motion; or v) any combination of i-iv.

In some cases, such as when the biometric sensor data includes PPG data, extracting the physiological information at block 704 includes determining a peripheral arterial tone (PAT) signal (e.g., from the PPG data). In some cases, the PAT signal can be used to derive further physiological information, such as i) respiration rate data, ii) heart rate data, iii) heart rate variability data, or iv) any combination of i-iii.

In some cases, such as when the biometric sensor data includes GSR data, extracting the physiological information can include using the GSR data as physiological information. In some cases, when the biometric sensor data includes blood pressure data, extracting the physiological information can include using the blood pressure data as physiological information.

In some cases, extracting the physiological information at block 704 can include extracting a sympathetic nervous system (SNS) activation level and/or a parasympathetic nervous system (PNS) activation level.

In some cases, extracting the physiological information at block 704 can include extracting heart rate variability (HRV) data. In some cases, extracting a SNS activation level or a PNS activation level can be based at least in part on HRV data. In some cases, extracting the SNS activation level or the PNS activation level can be based at least in part on a power spectral density of the HRV data. In some cases, extracting the SNS activation level or the PNS activation level can be based at least in part on an analysis of high frequency components of the HRV data. In some cases, an analysis of the high frequency components of the HRV can be used to determine an index of PNS activity. Low frequency is often defined as around 0.04-0.15 Hz, whereas High frequency is around 0.15-0.4 Hz, although that need not always be the case

In some cases, the HRV may be greater in those that are healthy and lower in those diagnosed with OSA (especially those with AHI >30) and not treated with PAP. Adherent and correctly set up PAP users may have HRV similar to their healthy state (for a similar age, gender, BMI, no comorbidities etc.).

In some cases physiological information can include a breathing or respiration signal and/or associated features, such as i) variability of breathing rate throughout the day and/or night, which can be characteristic of the individual, and which can be inter-breath and/or over longer timescales (e.g., 30, 60, 90 seconds, or much longer); ii) the stability of the breathing rate over time; iii) the standard deviation of breathing rate; iv) the depth of respiration (e.g., shallow, deep, etc.), and/or the relative amplitude of adjacent breaths; v) the mean or average value of the breathing rate; vi) the trimmed mean (e.g., at 10%) of the breathing rate to reject outliers; vii) a wake or asleep state; viii) surges (sudden accelerations or decelerations) in breathing rate (e.g., as seen during quiet periods and during REM sleep); ix) median (50th percentile) of the breathing rate; x) interquartile range (25th-75th Percentile) of the breathing rate; xi)_5th-95th percentile of the breathing rate; xii) 10th-90th percentile of the breathing rate; xiii) shape of the breathing rate Histogram; xiv) skewness of the breathing rate; xv) kurtosis of the breathing rate; xvi) peak Frequency of the breathing rate over time; xvii) ratio of second and third harmonics of peak frequency of the breathing rate; xviii) percentage of valid data (e.g., valid physiologically plausible data) in a breathing signal; xix) autocorrelation of the individual signals; xx) characteristic patterns in the spectrogram of the breathing rate; and xxi) relative percentage of REM and deep sleep.

In some cases, physiological information can include cardiac (heart) signals and associated features, such as i) heart rate variability HRV (inter beat (e.g., as derived from the Ballistocardiogram) and over longer defined moving windows—e.g., 30, 60, 90 sec); ii) variability over time (interbeat/breath variability)); iii) mean; iv) trimmed mean (10%); standard deviation; v) median (50th percentile); vi) interquartile range (25th-75th percentile); vii) 5th-95th percentile; viii) 10th-90th percentile; ix) shape of the cardiac signal histogram; x) skewness of the cardiac signal; xi) kurtosis of the cardiac signal; xii) stability over time of the cardiac signal; xiii) peak frequency over time of the cardiac signal; xiv) ratio of second and third harmonics of peak frequency of the cardiac signal; xv) percentage of valid data (e.g., valid physiologically plausible data); xvi) a wake or asleep state; xvii) autocorrelation of the individual signals; and xviii) characteristic patterns in the spectrogram of the cardiac signal.

In some cased, physiological information can include cardiorespiratory signals. Examples of such signals include i) magnitude square cross spectral density (e.g., in a moving window); ii) cross coherence; iii) respiratory sinus arrhythmia peak; iv) low frequency (LF) over high frequency (HF) ratio to indicate autonomic nervous system parasympathetic/sympathetic balance; v) the cross correlation, cross coherence (or cross spectral density) of the heart and breathing signal estimates; vi) non-linear estimates such as entropy measures; vii) the characteristic movement patterns over longer time scales (e.g., the statistical behavior observed in the signals); and viii) patterns of movement during detection of and comparison of these heart and breathing signals (e.g., during sleep, some people may have more restful and some more restless sleep).

At block 706, a stress level can be determined from the physiological information of block 704. Determining the stress level can include calculating the stress level using one or more pieces of the physiological information. In some cases, the stress level corresponds to a discrete physiological information value and/or a range of physiological information values.

The stress level from block 706 can be associated with a first period of time, which can be before, during, or after a sleep session of the user.

In some cases, determining the stress level at block 706 can include determining the stress level using a comparison of the SNS activation level and a baseline sympathetic activation level. In such cases, a baseline sympathetic activation level can be determined at block 714 from previous biometric sensor data received at block 712. Such previous biometric sensor data can be associated with a period of time prior to the period of time associated with the biometric sensor data from block 702. For example, the baseline sympathetic activation level can be based on a time period preceding a sleep session, over a number of such time periods, over a number of sleep sessions, or the like.

At block 708, a target stress level is determined. Determining a target stress level at block 708 can occur similarly to determining a target physiological parameter at block 506 of FIG. 5.

At block 710, the entrainment program can be presented. Presenting the entrainment program at block 710 can be similar to presenting the entrainment program at block 518 of FIG. 5, except based specifically on stress levels instead of physiological parameters. For example, presenting the entrainment program at block 710 can include determining an entrainment signal based at least in part on the stress level and the target stress level, and presenting the entrainment stimulus to the user based at least in part on the entrainment signal. In some cases, an achievement indicator can also be optionally presented.

In some optional cases, at block 726, the stress level from block 706 can be compared with an additional stress level to determine an efficacy metric (e.g., efficacy level) of the entrainment program. While the stress level from block 706 is associated with a first time period, the additional stress level can be similarly obtained, but associated with a second time period. The first time period and second time period can occur before, during, or after an entrainment program has been presented such that an efficacy metric of the entrainment program can be determined (e.g., if the stress level decreases after the entrainment program, the entrainment program can be considered effective, optionally with a value corresponding to the amount of decrease in stress level). Thus, if the second time period occurs after the first time period, i) the first time occurs before presenting the entrainment program and the second time occurs after presenting the entrainment program; or ii) at least one of the first time and the second time occur during presenting the entrainment program. In some cases, the first time period and second time period can be continuous, or otherwise part of the same process, such that the additional received sensor data that is used to determine the additional stress level can be received during a subsequent iteration of block 702.

In some optional cases, the stress level from block 706 can be compared to one or more threshold values at block 716 to determine whether or not the stress level is outside of the threshold(s). The stress level is outside of the threshold(s) when the stress level exceeds a maximum threshold level and/or the stress level falls below a minimum threshold level, optionally for a threshold duration of time. If the stress level is outside of the threshold(s), it can trigger an action to be performed at block 718. Any suitable action can be performed based on the stress level being outside of the threshold(s).

In some cases, performing an action at block 718 includes presenting a notification at block 720. Presenting the notification can include presenting a visual, aural, tactile, or other stimulus to the user and/or a caregiver associated with the user. For example, such a notification can indicate that the user's stress level is especially high, requiring action by the user and/or caregiver associated with the user.

In some cases, performing an action at block 718 includes generating a report at block 722. Generating the report can include generating an indication that a particular diagnostic test (e.g., a screening test) that was undertaken at a time period associated with the stress level (e.g., the time period during which the biometric sensor data from block 702 was acquired) is invalid or likely to be invalid. For example, when the system determines that the user's stress level during a particular sleep session was too high (e.g., exceeded a predetermined threshold, such a predetermined respiration rate (or HR, HRV, or sympathetic nervous system activation, etc.) for a specified duration of time), the system may generate a report indicative that the home sleep test being conducted by the user during that sleep session is likely to be invalid due to the user's high stress levels. Generating the report at block 722 can include presenting a notification of the report similarly to presenting the notification at block 720.

In some cases, performing an action at block 718 can include adjusting presentation of the entrainment program at block 724. Adjusting presentation of the entrainment program can include adjusting the entrainment signal (e.g., modifying the determined entrainment signal or affecting determination of the entrainment signal) and/or adjusting presentation of the entrainment stimulus.

Determining that the stress level is outside of threshold(s) and then performing the action in response can provide multiple benefits, such as i) notifying the user or a caregiver of the high/low stress level of the user; ii) providing an indication when diagnostic tests may be invalid; and iii) attempting to improve the user's stress level when it is so far out of threshold(s).

In some cases, process 700 can repeat by continuing to receive biometric sensor data at block 702. While the blocks of process 700 are depicted in a certain order, some blocks can be removed, new blocks can be added, and/or blocks can be moved around and performed in other orders, as appropriate. For example, in some cases, determining the target stress level at block 708 and presenting the entrainment program at block 710 may occur only after determining that the stress level is outside of threshold(s) at block 716.

Similar to as described above with reference to entrainment programs associated with respiratory signals, an entrainment program associated with stress level (e.g., with the goal of moving the user's current stress level to a target stress level) can be monitored for efficacy and can be trained for a given user. Such training can occur over the course of one or more previous instances of using the entrainment program. Such previous instances can include times during preceding days, previous sleep sessions, previous HSAT test sessions, and the like. The results of such previous instances can be used to tailor future instances. For example, if a particular entrainment program was initially ineffective at reducing stress levels, but became more effective when a certain parameter of the entrainment signal was adjusted, the system may use that information to further adjust the certain parameter to try and achieve even more effective reduction of stress levels.

One or more elements or aspects or steps, or any portion(s) thereof, from one or more of any of claims 1 to 60 below can be combined with one or more elements or aspects or steps, or any portion(s) thereof, from one or more of any of the other claims 1 to 60 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving biometric sensor data associated with a user;
extracting respiration information from the biometric sensor data;
determining a target respiration pattern; and
presenting an entrainment program to the user based at least in part on the target respiration pattern, wherein presenting the entrainment program facilitates entraining a respiration pattern of the user towards the target respiration pattern, and wherein presenting the entrainment program includes:
determining an entrainment signal based at least in part on the respiration information and the target respiration pattern; and
presenting an entrainment stimulus to the user based at least in part on the entrainment signal.

2-5. (canceled)

6. The method of claim 1, further comprising receiving historical entrainment efficacy information associated with the user, wherein the historical entrainment efficacy information is indicative of the efficacy of presentation of one or more prior entrainment stimuli, and wherein presenting the entrainment program is further based at least in part on the historical entrainment efficacy information.

7. The method of claim 1, wherein presenting the entrainment stimulus includes i) generating an audio stimulus; ii) modulating an existing audio stimulus; iii) generating a visual stimulus; iv) modulating an existing visual stimulus; v) generating a tactile stimulus; vi) modulating an existing tactile stimulus; vii) adjusting an airflow setting of a respiratory device fluidly coupled to a respiratory system of the user; viii) or any combination of i-vii.

8. The method of claim 1, wherein receiving the biometric sensor data occurs while the user is asleep, and wherein presenting the entrainment program to the user occurs at least in part while the user is asleep, and wherein determining the target respiration pattern includes:
determining a sleep stage of the user from the biometric sensor data and a desired sleep stage of the user;
determining that the determined sleep stage of the user is different than the desired sleep stage of the user; and
setting the target respiration pattern to a respiration pattern associated with the desired sleep stage of the user.

9-11. (canceled)

12. The method of claim 1, wherein extracting respiration information from the biometric sensor data includes determining a breath path parameter indicative of the user engaging in nasal breathing or mouth breathing, and wherein presenting the entrainment program includes presenting a breath path stimulus based at least in part on the breath path parameter to facilitate inducing the user to engage in nasal breathing.

13. The method of claim 1, further comprising:
generating a first comfort score based at least in part on the biometric sensor data, wherein the first comfort score is indicative of a first subjective comfort level of the user before presenting the entrainment stimulus;
receiving additional biometric sensor data associated with the user after initially presenting the entrainment program;
generating a second comfort score based at least in part on the additional biometric sensor data, wherein the second comfort score is indicative of a second subjective comfort level of the user during or after presenting the entrainment stimulus; and
determining an entrainment comfort score based at least in part on a change between the first comfort score and the second comfort score.

14. The method of claim 1, further comprising:
receiving additional biometric sensor data associated with the user after initially presenting the entrainment program;
generating an entrainment persistence score based at least in part on the biometric sensor data and the additional biometric sensor data, wherein the entrainment persistence score is based at least in part on i) a difference between a respiration pattern of the user and the target respiration pattern, ii) a rate of change of the respiration pattern, iii) a length of time the respiration pattern remains within a threshold of the target respiration pattern, iv) a length of time the rate of change of the respiration pattern remains within a rate of change threshold, or v) any combination of i-iv;
extracting sleep quality information from the additional biometric sensor data, wherein the additional biometric sensor data includes at least a portion of data collected during a sleep session; and
generating a respiratory therapy compliance score based at least in part on the entrainment persistence score and the sleep quality information, wherein the respiratory therapy compliance score is indicative of a likelihood to comply with a respiratory therapy program that includes use of a removable user interface to deliver respiratory therapy.

15. (canceled)

16. The method of claim 14, wherein receiving the biometric sensor data occurs during an entrainment session prior to a sleep session, wherein receiving the additional biometric sensor data occurs during at least the sleep session; and wherein the method further comprises:
generating a sleep score associated with the sleep session based at least in part on the additional biometric sensor data;
generating an entrainment effectivity score based at least in part on the entrainment persistence score and the sleep score, wherein the entrainment effectivity score is indicative of a correlation between the entrainment persistence score and the sleep score; and
presenting the entrainment effectivity score.

17. (canceled)

18. The method of claim 1, wherein presenting the entrainment stimulus includes adjusting one or more parameters of a respiratory therapy device according to the entrainment signal.

19. The method of claim 1, wherein the respiration information includes i) respiration rate; ii) time between breaths; iii) a time of maximal inspiration; iv) a time of maximal expiration; v) respiration rate variability; vi) respiration morphology information; vii) a current inspiration rate, viii) a current expiration rate, ix) a lung capacity parameter, or x) any combination of i-ix.

20-22. (canceled)

23. The method of claim 1, further comprising:
receiving additional biometric sensor data associated with the user;
extracting a sleep onset latency from the additional biometric sensor data; and
in response to determining that the sleep onset latency exceeds a threshold, presenting the entrainment program or presenting a suggestion to begin the entrainment program.

24-25. (canceled)

26. The method of claim 1, wherein presenting the entrainment program further includes presenting an achievement indicator indicative of a progression of the respiration pattern of the user towards or away from the target respiration pattern.

27-29. (canceled)

30. The method of claim 18, wherein receiving the biometric sensor data occurs while the user is receiving respiratory therapy from a respiratory therapy device.

31-32. (canceled)

33. A method comprising:
receiving biometric sensor data associated with a user;
extracting physiological information indicative of a stress level from the biometric sensor data;
determining the stress level from the extracted physiological information;
determining a target stress level; and
presenting an entrainment program to the user based at least in part on the target stress level, wherein presenting the entrainment program facilitates modifying a stress level of the user towards the target stress level, and wherein presenting the entrainment program includes:
determining an entrainment signal based at least in part on the stress level and the target stress level; and
presenting an entrainment stimulus to the user based at least in part on the entrainment signal.

34. The method of claim 33, wherein:
the physiological information indicative of the stress level includes i) a respiration rate, ii) heart rate, iii) heart rate variability, iv) user motion; or v) any combination of i-iv; and
the biometric sensor data includes galvanic skin response (GSR) data acquired by a GSR sensor, photoplethysmography (PPG) data acquired by a PPG sensor, blood pressure data acquired by a blood pressure monitor, electroencephalogram (EEG) data acquired from an EEG sensor, electrocardiogram (ECG) data acquired from an ECG sensor, or any combination thereof

35-45. (canceled)

46. The method of claim 33, wherein extracting the physiological information indicative of the stress level includes extracting at least one of a sympathetic nervous system activation level and a parasympathetic nervous system activation level, wherein presenting the entrainment program occurs during a sleep session of the user, and wherein the method further comprises:
receiving previous biometric sensor data associated with the user prior to the sleep session; and
determining a baseline sympathetic activation level from the previous biometric sensor data;
wherein determining the stress level from the extracted physiological information includes comparing the sympathetic nervous system activation level to the baseline sympathetic activation level.

47. (canceled)

48. The method of claim 46, wherein determining the entrainment signal includes determining a desired entrainment signal that i) depresses the sympathetic nervous system activation level of the user, ii) stimulates the parasympathetic nervous system activation level of the user, or iii) both i) and ii).

49-53. (canceled)

54. The method of claim 33, further comprising:
determining that the stress level, for a predetermined portion of a sleep session, exceeds a maximum threshold stress level or falls below a minimum threshold stress level;
performing an action in response to determining that the stress level exceeds the maximum threshold stress level or falls below the minimum threshold stress level, wherein the action includes i) presenting a notification to the user; ii) presenting a notification to a caregiver associated with the user; iii) generating a report that a diagnostic test occurring during the sleep session is invalid; iv) adjusting the entrainment signal; v) adjusting presentation of the entrainment stimulus; or vi) any combination of i-v.

55. The method of claim 33, further comprising:
storing the stress level, wherein the stress level is associated with a first time;
determining an additional stress level based at least in part on additional biometric sensor data associated with the user at a second time after the first time, wherein i) the first time occurs before presenting the entrainment program and the second time occurs after presenting the entrainment program; or ii) at least one of the first time and the second time occur during presenting the entrainment program; and
determining an efficacy level of the entrainment program based at least in part on a comparison of the stress level and the additional stress level.

56. (canceled)

57. A system comprising:
a control system including one or more processors; and
a memory having stored thereon machine readable instructions;
wherein the control system is coupled to the memory, and wherein the one or more processors of the control system are configured to execute the machine readable instructions in the memory to:
receive biometric sensor data associated with a user;
extract respiration information from the biometric sensor data;
determine a target respiration pattern; and
present an entrainment program to the user based at least in part on the target respiration pattern, wherein presenting the entrainment program facilitates entraining a respiration pattern of the user towards the target respiration pattern, and wherein to present the entrainment program, the one or more processors of the control system are further configured to:
determine an entrainment signal based at least in part on the respiration information and the target respiration pattern; and
present an entrainment stimulus to the user based at least in part on the entrainment signal

58-60. (canceled)
